(19) United States
(12) Patent Application Publication
(10) Pub. No.: US 2002/0172683 A1
(43) Pub. Date: Nov. 21, 2002

(54) MHC-I-restricted presentation of HIV-1 virion antigens without viral replication. Application to the stimulation of CTL and vaccination in vivo; analysis of vaccinating composition in vitro

(76) Inventors: Olivier Schwartz, Paris (FR); Florence Buseyne, Paris (FR); Delphine Marsac, Paris (FR); Marie-Louise Michel, Paris (FR); Yves Riviere, Paris (FR); Jean-Michel Heard, Paris (FR)

Correspondence Address: Finnegan, Henderson, Farabow,, Garrett & Dunner, L.L.P., 1300 I Street, N.W., Washington, DC, 20005-3315, US

(21) Appl. No.: 10/083,678

(22) Filed: Feb. 27, 2002

Related U.S. Application Data
(60) Provisional application No. 60/271,432, filed on Feb. 27, 2001.

(51) Int. Cl. A61K 39/12; C12N 7/00; C12N 15/86

(52) U.S. Cl. 424/186.1; 435/320.1; 435/235.1; 435/456

(57) ABSTRACT

Dendritic cells and macrophages can process extracellular antigens for presentation by MHC-I molecules. HIV-1 epitopes derived from incoming virions are presented through the exogenous MHC-I pathway in primary human dendritic cells, and to a lower extent in macrophages, leading to cytotoxic T lymphocyte activation in the absence of viral protein neosynthesis. Exogenous antigen presentation required adequate virus-receptor interactions and fusion of viral and cellular membranes. These results provide new insights about how anti-HIV cytotoxic T lymphocytes can be activated and are useful for anti-HIV vaccine design.

### CROSS-REFERENCE TO RELATED APPLICATIONS

This application is based on and claims the benefit of U.S. Provisional Application Ser. No. 60/271,432, filed Feb. 27, 2001 (attorney docket no. 03495.6064) The entire disclosure of this application is relied upon and incorporated by reference herein.

### BACKGROUND OF THE INVENTION

This invention relates to immunogenic compositions, including vaccinating compositions, and their use for the treatment of viral pathologies, such as those due to HIV-1 or HIV-2 infections. In addition, this invention relates to the stimulation of cytotoxic lymphocytes through an MHC-I restricted exogenous antigen presentation pathway. Further, this invention relates to a process of screening compounds and compositions useful in the treatment and/or prevention of such viral pathologies.

CD8+ cytotoxic T lymphocytes (CTLs) kill cells infected with intracellular pathogens, such as viruses, parasites, or bacteria. CTLs recognize specific peptides borne by major histocompatibility complex class I (MHC-I) molecules. In most cells, MHC-I molecules associate exclusively with peptides derived from neo-synthesized proteins. Extracellular antigens are usually not processed for presentation by MHC-I molecules, thus avoiding CTL killing of cells that may have internalized antigens from infected or tumor cells. In contrast, professional antigen presenting cells (APCs), such as dendritic cells (DCs), macrophages, and B cells, have the capacity to process antigens from extracellular sources for presentation on MHC-I molecules. This alternative “exogenous” pathway, also referred to as “cross-presentation”, most likely plays an important role in the generation of CTL immunity ¹⁻³.

APCs capture exogenous antigens through multiple pathways, including non-specific mechanisms, such as phagocytosis or macropinocytosis, as well as specific receptor-mediated delivery pathways ^{1,3,4}. Capture pathways influence the efficiency of antigen processing and presentation. High antigen concentrations are required for CTL activation following macropinocytosis or phagocytosis of soluble antigens, raising questions about the in vivo relevance of these uptake pathways ⁵. Antigen aggregation, coupling with beads or association with heat shock proteins strongly enhances presentation efficiency ⁶⁻⁸ Internalization of antigens through specific membrane receptors, such as Fc_ or mannose receptors, also results in efficient MHC-I restricted antigen presentation ^{4,9}. Processing and MHC-I presentation of captured antigens often occur via the classical, cytosolic proteasome- and TAP-dependent pathway. Reaching this pathway necessitates specific routes for antigen delivery from endosomes or phagosomes to the cytosol ^{1,10}. Alternatively, captured antigens may be directly processed in a non-cytosolic pathway, in intracellular vesicles, or at the plasma membrane ^{1,8,11}.

DCs are the only APCs that can stimulate resting naive T lymphocytes and initiate CTL responses ¹². Immature DCs residing in peripheral tissues capture antigens from various sources, including microbes and infected cells, cell debris, proteins, and immune complexes. Antigen-loaded DCs travel toward secondary lymphoid organs, processing antigens for presentation, and acquiring the capacity to attract and activate resting CD8+ CTLs during that journey. The presentation of exogenous antigens by DCs is required for the stimulation of CTLs against transplants, tumors, bacteria, or antigens from viruses that do not infect APCs ^{3,13,14} However, direct evidence for the induction of CTLs against viral antigens through the exogenous pathway during viral infection is sparse. A number of in vitro observations has been made in experimental systems using immortalized fibroblasts or lymphocytes as stimulators ^{3,15,16}. It is unknown how representative these studies are for professional APCs. In vivo, a role for cross-presentation has been established in a mouse model of poliovirus infection. Induction of CTL immunity to this virus, which does not replicate in APCs, requires exogenous presentation of viral antigens ¹⁴.

Whether the exogenous MHC-I-restricted presentation pathways play a role during human viral infections remains unclear. The question is especially relevant to HIV-1 infection, since DCs and macrophages are natural targets of HIV-1 infection. These cells play a crucial role in virus transmission and propagation. Immature DCs residing in the skin and mucosa are thought to be the first cell targets of HIV-1. DCs transport virus particles and transmit a vigourous infection to T cells in lymph nodes ^{17,18}. DCs express low levels of the HIV receptor CD4 and coreceptors CCR5 or CXCR4. Although the virus replicates rather inefficiently in these cells, both R5- and X4-tropic HIV-1 readily bind and enter DCs ¹⁹-22. Transmission of HIV-1 from DCs to T cells involves a specific DC receptor, DC-SIGN, which binds the viral envelope protein and retains the attached virus in an infectious state ²³. Macrophages also express receptors for HIV-1. However, replication is restricted to R5-tropic strains, perhaps because the CXCR4 co-receptor has a reduced ability to support viral entry ²⁴.

There exists a need in the art for compounds, compositions, and methods for the treatment and/or prevention of viral infections, such as infections by HIV-1. The compounds, compositions, and methods should be useful in the development of immunogens for vaccines, such as vaccines based on attenuated or inactivated infectious viral particles or viral subunits.

### SUMMARY OF THE INVENTION

This invention aids in fulfilling these needs in the art. The invention provides an immunogenic composition capable of inducing a cytotoxic response, more particularly a CTL cytotoxic response, in vitro or in vivo against a viral disease through a MHC-I restricted exogenous antigen presentation pathway without requiring viral replication. The immunogenic composition contains at least one of:

(A) a first plasmid containing a polynucleotide corresponding to the entire or a part of the viral genome and a second plasmid comprising in an insert containing a polynucleotide coding for a viral envelope (a part of the envelope or a surface protein) and being under the control of a promoter, said plasmids being selected for their fusogenic properties when binding to antigen presentation cells, and for inducing a cytotoxic response through a MHC-1 restricted exogenous antigen presentation pathway;

(B) a virus with intact fusogenic capacities, but whose infectious capacities have been inactivated or attenuated; and

(C) viral particles obtained by the purification of a cell culture supernatant.

The viral particles obtained by the purification of a cell culture supernatant can be prepared by transfecting producing cells, for example, Hela (35) or 293 (27), with the plasmids and purifying the supernatant, or by infecting antigen presenting cells with an HIV virus, purifying the supernatant, and inactivating or attenuating the infectious capacity of the virus. The vaccinating composition can be combined with a pharmaceutically acceptable vehicle or another vaccine.

This invention also provides a process of treatment of a host suffering from a viral pathology comprising administering a plasmid comprising a polynucleotide coding for the entire or a part of a virus genome and containing an insert comprising a polynucleotide coding for a viral envelope (or a part of the envelope or a surface protein), and being under the control of a promoter. The plasmid is selected for its fusogenic, non-replicative properties, and for inducing a cytotoxic response after a MHC-I restricted exogenous antigen presentation.

The virus can be a human or animal retrovirus, such as HIV-1, HIV-2, SIV, FeLV, or FIV. The host can be a mammal, such as a human or a mouse.

In another embodiment of the invention, a process of stimulation in vivo of cytotoxic lymphocytes through an MHC-I restricted exogenous antigen presentation pathway without requiring viral replication, comprises:

(A) administering the plasmids contained in the immunogenic composition according to the invention to the host; or

(B) optionally testing the cytotoxic T cells after the step (A) above in a cytotoxic test comprising incubating an organ or a biologic fluid of a host containing cytotoxic T cells of the host with a synthetic peptide encoded by a viral genome contained partly in the first or the second plasmid or using target cells with the same HLA haplotype as the host or a compatible HLA haplotype, wherein the target cell is incubated with a synthetic peptide encoded by an HIV- genome contained in the first or second plasmids.

Alternatively, viral particles obtained by supernatant purification can be employed. In another embodiment an HIV virus whose infectious capacities have been inactivated or attenuated, but whose fusogenic capacities are intact, is employed.

Antigen presenting cells can be treated with the immunogenic composition according to the invention and then administrated back to the mammal after incubation.

In addition, this invention provides a process of screening a composition, which is capable of inducing against a viral pathology a cytotoxic response in vitro or in vivo by exogenous antigen presentation without viral replication.

In an exemplary embodiment, this invention examines whether antigens brought by incoming HIV-1 virions are processed for CTL presentation in APCs. This invention demonstrates that HIV-1 epitopes are presented by MHC-I, in the absence of viral protein neosynthesis, in primary human DCs and to a lower extent in macrophages, but not in CD4+ lymphocytes. Exogenous presentation required interactions between viral envelope glycoproteins and their receptors, as well as the fusogenic activity of the viral envelope. Exogenous presentation may play a key role in the triggering of an anti-HIV-1 CTL not only in seropositive individuals, but also in HIV-resistant persons at high risk for infection.

### BRIEF DESCRIPTION OF THE DRAWINGS

This invention will be described in detail with reference to the drawings in which:

FIG. 1 shows the results of MHC-I presentation of a Gag p17 epitope derived from incoming HIV-1 virions.

Primary immature DCs (A), macrophages (B) and CD4+ lymphocytes (C) prepared from HLA-A2+ HIV-seronegative individuals and B lymphoblastoid cells expressing HLA A2 (C1R-A2) (D) were used as stimulator cells in an IFN-γ-Elispot assay. The effector was the CD8+ CTL line EM71-1, which recognizes an HLA-A2-restricted epitope (SL9) from the Gag p17 protein. Stimulating cells were pretreated with AZT, exposed to the indicated viruses, and incubated with EM71-1 cells. Activity of EM71-1 cells is depicted as the number of IFN-γ positive cells for 1000 effector. As a positive control, stimulating cells were pulsed with the SL9 peptide. HIV_{BRU}(VSV) is an env-deleted HIV-1 pseudotyped with the VSV-G envelope. Data are mean ±s.d. of duplicates and are representative of at least 3 independent experiments.

FIG. 2 shows the characteristics of exogenous presentation of HIV-1 antigens by MHC-I.

Panel (A) shows exogenous presentation of Gag epitopes is envelope-dependent and occurs with HIV-vector particles. HLA A2+ DCs and macrophages were pretreated with AZT, exposed to the indicated virus and an Elispot assay was performed using EM71-1 effectors. EM71-1 cells do not recognize target cells exposed to env-deleted HIV (HIV_{BRU}Δenv). In contrast, HIV-vector particles, which do not encode HIV proteins but carry a functional VSV-G envelope, activate effector cells.

Panel (B) shows that aldrithiol-2 (AT-2) inactivated HIV-1 virions are processed for MHC-I restricted exogenous presentation. Exposure of DCs to AT-2-inactivated HIV_{MN} strain induces IFN-γ production by EM71-1 cells effectors as efficiently as exposure to infectious HIV_{MN}.

Panel (C) shows that exogenous presentation of HIV-1 Gag epitopes requires fusion of viral and cellular membranes. Exogenous HIV presentation is not observed with viral particles pseudotyped with fusion-defective VSV-G (mutant Q117N) or HIV-1 (mutant F522Y) envelopes. HIV_{BRU}(HIV) and HIV_{BRU}(HIV_{F522}Y) are env-deleted HIV coated with a wild-type or a F522Y mutant HIV-1 envelope (from the X4-tropic HIV-1 strain HXB2), respectively. Data are mean ±s.d. of duplicates and are representative of 2-3 independent experiments.

FIG. 3 shows MHC-I presentation of a Gag p24 epitope derived from incoming HIV-1 virions.

B lymphoblastoid cells expressing HLA B53 (C1R-B53) were used as targets in a standard ⁵¹Cr-release assay. The effector was the CD8+ CTL clone 141, which recognizes an HLA-B53-restricted epitope (QW9) from the Gag p24 protein. C1R-B53 cells were pretreated with AZT and exposed to the indicated viruses, or pulsed with the cognate peptide QW9 before ⁵¹Cr-release assay. HIV_{NL43}Δenv is an env-deleted virus. HIV_{NL43}(VSV) is an env-deleted virus pseudotyped with the VSV-G envelope. HIV-vector particles are HIV-1 virions containing Gag and Pol-derived proteins coated with a VSV-G envelope. Vector genome does not encode HIV-1 proteins. Data are mean ±s.d. of triplicates for ⁵¹Cr-release assays and are representative of 3 independent experiments. E/T: Effector/Target ratio.

FIG. 4 shows the results of analysis of CTL response to incoming HIV-1 virions.

Panel (A) shows that CTL response is MHC-I-restricted. Indicated B-EBV transformed cells, expressing or not HLA-B53, were pulsed with the cognate peptide QW9 (left panel), or were pretreated with AZT and exposed to HIV_{NL43}(VSV) (500 ng of p24 for 10⁶ cells) (right panel). Cells were used as targets in a standard ⁵¹Cr-release assay. The effector is the HLA-B53-restricted CTL clone 141 described in FIG. 3.

Panel (B) shows the kinetics of CTL response. C1R-B53 were pretreated with AZT and exposed to HIV_{NL43}(VSV). Cells were then incubated for the indicated periods of time at 37° C. in the presence of AZT and assayed with CTL clone 141 as effector. Background killing of uninfected cells was below 3%. Lysis of QW9-pulsed cells was 70%.

Panel (C) is a dose-response analysis of CTL activity. C1R-B53 were pretreated with AZT (5 μM) for 2 h and exposed to the indicated amounts of HIV_{NL43}(VSV). Cells were then assayed using CTL clone 141 as effector. Lysis of QW9-pulsed cells was 70%. E/T ratio: 10/1. Data are mean ±s.d. of triplicates and are representative of 3 independent experiments.

FIG. 5: Efficiency of the Gag-specific cytotoxic T cell response after DNA-coinjection. Mice were immunized with 10 μg (left panel) or 100 μg (right panel) of pCMV.ΔR8-2+pCMV.AS (open diamond) or pCMV.ΔR8-2+pCMV.VSV (black square) plasmids DNA encoding “naked” or VSV-G-pseudotyped Gag particles, respectively. DNA was injected into normal muscle. Cytotoxic activity of in vitro stimulated spleen T cells was measured 2 weeks after immunization. The specific lysis was calculated by subtracting the non-specific lysis on P815 target cells from the specific lysis obtained on P815 cells pulsed with HIV-1 p24 gag peptide. Specific lysis values represent mean values +/− SEM from five individual mice in each immunization group.

FIG. 6: Dose-dependent cytotoxic T cell responses after co-injection of DNAs coding for “naked” or VSV-G-pseudotyped Gag particles. Mice were immunized with 1, 10 or 100 μg of either pCMV.AR8-2+pCMV.AS (open columns) or pCMV.ΔR8-2+pCMV.VSV (filled columns) plasmid DNA. DNA was injected into either normal muscle (left panel) or cardiotoxin-pretreated muscle (regenerating muscle, right panel). Cytotoxic activity of spleen cells was measured using peptide-loaded or unloaded P815 cells as targets. Cytolytic responses were considered positive after substraction of the background when the specific lysis was 10% or more at an effector to target ratio of 100/1. Number of responding mice on tested mice is indicated at the top of each column and represents cumulative results obtained from three to five independent experiments. * p<0.05, ** p<0.001 by the χ² Pearson test.

FIG. 7: Analysis of in vitro processing of Gag particles. An IFN-γ-Elispot assay was performed with Gag-specific effector T cells obtained from mice immunized with pCMV.ΔR8-2 DNA encoding “naked” Gag particles. The number of IFN-γ spot-forming cells (IFN-γ-SFC) per 10⁶ splenocytes was measured in response to a short-term stimulation of splenocytes (40 h) with either naked or VSV-G-pseudotyped Gag particles. The number of specific SFC was calculated after substracting the background obtained in wells containing splenocytes in culture medium. Different concentrations of viral particles were tested for their ability to present Gag epitopes (100, 20 and 4 ng/ml HIV-1-p24). Results are mean values±SEM from three individual mice. Note that the number of IFN-γ-SFC is expressed per 10⁶ splenocytes.

FIG. 8: Analysis of T cell sub-populations activated after in vitro processing of Gag particles. IFN-γ-Elispot assay was performed as in FIG. 7. Effector T cells were pooled splenocytes from five mice immunized with pCMV.ΔR8-2 DNA encoding “naked” Gag particles. The number of Gag-specific IFN-γ spot forming cells (IFN-γ-SFC) was measured in response to a short-term stimulation of the splenocytes with HIV-1 Gag peptide (1 μg/ml), VSV-G-pseudotyped HIV-1 Gag particles (p24, 100 ng/ml) or “naked” HIV-1 Gag particles (p24, 100 ng/ml). ELISPOT assay was performed on undepleted (A), CD4⁺ T cell-depleted (B) and CD8⁺ T cell-depleted (C) splenocytes. Note that IFN-γ SFC are expressed for respectively 10⁶ T lymphocytes (A), 10⁶ CD8⁺ T cells (B) and 10⁶ CD4⁺ T cells (C) after staining and quantification of each cell population by FACS analysis.

FIG. 9. CD4⁺ T cell responses induced in vivo by injection of DNAs encoding “naked” or VSV-G-pseudotyped particles. Groups of 5 or 11 mice were injected with 100 μg of DNA vectors encoding either “naked” (pCMV.ΔR8-2+pCMV.AS) or VSV-G-pseudotyped Gag particles (pCMV.ΔR8-2+pCMV.VSV) into normal muscle (left panel) or in regenerating muscle (right panel). Two weeks after DNA-immunization, ex vivo Elispot assay was performed on splenocytes to measured Gag-specific IFN-γ secreting CD4⁺ T cells. Splenocytes were incubated 40 hours with “naked” Gag particle (100 ng/ml) or in culture medium. Results were given as mean of number of specific IFN-γ secreting CD4+ T cells per spleen ±SEM.

### DETAILED DESCRIPTION OF THE INVENTION

In most cell types, peptides presented by MHC-1 are derived from endogenously synthesized proteins. In professional antigen presenting cells (APCs), such as dendritic cells (DCs), macrophages, and B lymphocytes, evidence exists for an additional MHC-I restricted pathway presenting peptides from extracellular origin. DCs and macrophages are two major targets of HIV replication and play a crucial role in transmission and propagation of viral infection.

This invention involves a study of the mechanisms of generation of MHC-I restricted HIV-1 epitopes in APCs. It was sought to be determined whether epitope generation requires de novo synthesis of HIV-1 protein or if alternatively incoming virions are considered as antigens and directly processed. This invention shows that epitopes from incoming HIV-1 virions are presented through the exogenous pathway of presentation in APCs, leading to CD8+ T lymphocyte activation in the absence of viral protein neosynthesis. MHC-1 restricted exogenous presentation occured efficiently in immature DCs with both CXCR4- and CCR5-tropic viruses as well as with HIV(VSV) pseudotypes. The phenomenon was less efficient in macrophages than in DCs and was not detected in CD4+ lymphocytes. In B cells, which lack HIV receptor CD4, MHC-1 restricted exogenous presentation was observed with HIV(VSV) pseudotypes only.

This invention shows that exogenous antigen presentation requires interaction between the viral envelope protein and its receptors and fusion of viral and cellular membranes. These results help explain how anti-HIV CTLs are activated and have implications for anti-HIV and other anti-viral vaccine design.

More particularly, this invention is the result of the discovery that primary APCs present MHC-I-restricted epitopes that had been generated from incoming HIV virions to specific CTLs. Presentation required fusion of virions with target cells and was observed in systems that precluded the possibility of de novo synthesis of viral proteins, confirming the exogenous nature of the antigen presented. HIV-vector particles, which do not encode for any viral protein, as well as AT-2-inactivated HIV-1 virions, whose infectivity is abrogated but are still able to enter target cells, efficiently activate CTLs after exposure to APCs. Exogenous presentation occurred with various epitopes (from Gag p24 and p17) and MHC-I molecules (HLA-A2 and HLA-B53). The phenomenon was particularly efficient in immature DCs, where it was observed with both R5 and X4-tropic HIV-1 strains, which entry is pH-independent, and with HIV(VSV) pseudotypes, which enter through a pH-dependent endocytic pathway. Envelope-free, as well as fusion-defective virions, did not stimulate effector cells, indicating that non-specific uptake of viral particles (i.e. by phagocytosis or macropinocytosis) does not lead to detectable exogenous presentation of HIV-1 epitopes. In contrast, the receptor-dependent uptake of HIV-1 virions in APCs induced a strong CTL activation. Therefore, as previously reported for Fc_or mannose receptors, the receptor-mediated internalization of antigens (whole virus particles in the Examples herein) induce an efficient cross-presentation ^{4,9}. This invention also shows that whatever the virion entry pathway (pH-dependent or -independent), fusion of viral and cellular membranes, and thus delivery of virion proteins to the cytosol, are required for exogenous presentation. The proteasome, which degrades HIV particles in vitro and incoming virions in HeLa cells 35, is therefore likely involved in the processing of viral epitopes. The toxicity of proteasome inhibitors for the various cells used here precluded direct investigation of this issue.

Exogenous presentation of HIV-1 antigens was less efficient in macrophages than in DCs. This may be the consequence of different patterns of HIV receptor expression (including CD4, chemokine receptors, or DC-SIGN) or different pathways of antigen uptake, intracellular transport, processing, and presentation to CTLs ^{3,10,23}. In CD4+ lymphocytes, exogenous presentation was not observed, although these cells express adequate HIV-1 receptors. This confirms that the ability to process exogenous antigens is likely to be restricted to professional APCs ^{1,3,4} In HIV-infected individuals, the absence of exogenous presentation in lymphocytes could prevent CTL-dependent killing of CD4+ cells that have been exposed to HIV proteins but are not productively infected.

In vivo evidence for a stimulation of CTLs by the exogenous pathway has been reported in a mouse model of viral infection ¹⁴. In vivo, APCs likely capture antigens from different origins, such as dying cells and debris, soluble or aggregated proteins and peptides, and immune-complexes. In the case of HIV infection, this demonstrates that an additional source of antigen is the virion itself, captured and delivered intracellularly through efficient envelope-receptor interactions. This invention reveals a novel aspect of the relationship between DCs and HIV-1. DCs are likely the first target cells encountered by the invading virus. DCs may take advantage of this early contact to process incoming HIV-1 particles through a fusion-dependent mechanism in order to trigger primary antiviral immunity. Accordingly, at least two lines of evidence suggest that the MHC-I-restricted exogenous pathway plays a key role in HIV-infected individuals. First, in professional APCs, presentation of epitopes before the synthesis of viral proteins may be essential for activation of CTLs, since Nef down-regulates MHC-I expression and decreases immune recognition of productively infected cells 36,37. Second, HIV-specific CTLs have been detected in highly-exposed but seronegative persons, including African sex-workers and regular partners of HIV-infected individuals without any evidence of viral replication ³⁸-40 This invention shows that in DCs, entry is the only step of the viral cycle required for CTL stimulation. It is, therefore, possible that in such HIV-resistant persons, the invading viral material may be processed for exogenous presentation in APCs and induce a CTL response without establishing productive infection.

This invention has clear implications for gene therapy and for anti-HIV-1 vaccine design. HIV-vectors are promising tools for gene therapy, due to their ability to transduce non-dividing cells ²⁷. The findings of this invention show that these vectors activate specific anti-HIV CTLs indicating that an anti-HIV response is anticipated in patients receiving HIV-vector-mediated gene therapy.

Moreover, DCs have the unique ability to initiate a primary CTL response in vivo ¹². A better understanding of HIV-1 interaction with DC provides new insights for manipulating the immune response enabling the design of new vaccination strategies. In HIV-infected individuals, CTLs are major contributors to antiviral immunity 40,41. In HIV-resistant persons, virus-specific CD8+ T cell responses in the absence of detectable HIV infection may play an important part in protective immunity against virus transmission ³⁸-40. Various anti-HIV vaccine attempts are, therefore, focused on generating CTL responses. This invention indicates that a low viral antigen input can be delivered in a manner that allows exogenous presentation, leading to the activation of specific CTLs. In particular, by using AT-2-inactivated HIV-1, this invention demonstrates that non-replicating viruses retaining a fusogenic potential are attractive as vaccines.

This invention has wide ranging implications for the treatment or prevention of viral infections including, but not limited to, HIV-1 and HIV-2. In brief, this invention shows that the anti-HIV-1 specific CTL response can be effectively activated without virus replication. This invention also shows that this exogenic presentation requires the receptor-dependent and fusion-dependent entry of viral material in the antigen-presenting cell. The scope of this invention will be more evident from the following definitions and discussion that follows.

The observation of the exogenous presentation of HIV-1 derived antigens has different applications, especially in the development of vaccine strategies that enable effective CTL induction.

As used herein, a “vaccine” is a preparation of at least one antigen that stimulates the development of antibodies or CTL in vivo, and thus confers active immunity against a specific disease or multiplicity of diseases.

This invention utlizes a “viral particle” that comprises a viral core and a viral envelope or another surface protein. The core can include the viral structural and immunogenic proteins. For retroviruses, the core can be composed of gag and pol gene products. The viral envelope glycoprotein, or another surface protein, can be a component of the viral membrane, which allows viral binding and entry into target cells, in the case of this invention, professional antigen presenting cells (APCs). The viral surface protein can be endogenous or exogenous to the wild type virus and is selected according to its ability to bind and to fuse with the membrane of APCs. Binding and entry are mediated or not by selected cellular receptors. In the case of retroviruses, the retroviral env gene product itself is an excellent candidate for mediating viral entry, since it will also act as an immunogenic component of the viral particle.

The viral particles are reproduced as closely as possible to wild-type viral particles, but are either attenuated or inactivated. This can be achieved by providing a plasmid or a viral vector carrying the set of nucleic acid sequences necessary for the reconstitution of the viral particle after expression in a cell in vitro. This can be accomplished by transfecting or transducing cells in vitro to produce the viral particles and then isolating or plurifying the particles for use.

The plasmid or viral vector carrying the set of sequences necessary for the reconstitution of a viral particular after expression in a host cell in vitro can contain the gag gene or the gag and env genes or the gag, pol and env genes a retrovirus. Alternatively, the retroviral particles can be expressed from a plasmid or vector having the envelope proteins of the virus, but lacking the viral genome. The virus can comprise any one or more of the gag, pol, and env sequences, but not the encapsulation sequence and alternatively, mutations INT and/or RT leading to the reconstitution in the cultured cell of an empty particle or of the particle carrying a defective viral genome. It is particularly advantageous when the viral genome of the virus is that of a retrovirus and the coding vector contains at least one structural gene. In a preferred embodiment, a cell can be transfected or transduced with a plasmid or viral vector comprising the set of sequences necessary for the manufacture by the cell of an attenuated or inactived HIV-1 and/or HIV-2 virus.

An “attenuated virus” is a viral particle composed of viral components, but which does not have the ability to replicate efficiently in vivo or in vitro and to induce a pathogenic syndrome in vivo. An attenuated virus is still able to replicate at low levels.

An “inactivated virus” does not replicate in vivo or in vitro, and does not accomplish a full viral life cycle upon exposure to its target. An inactivated virus can result, for example, from a modification of a gene that allows infection only through contact, such as by the deletion of the extracellular part of the env gene so as to retain only the fusogenic transmembrane part. In some cases, such as inactivation by aldithriol-2 (AT-2), the inactivated HIV-1 virus retains conformational and functional integrity of the viral envelope. AT-2 inactivated virions bind to and fuse with target cells, but the viral life cycle is arrested before initiation of reverse transcription. An inactivated virus fully or partly retains its immunogenic structure.

Attenuation or inactivation can be achieved (i) by introducing selected mutations or deletions in the viral genome, and/or (ii) by chemical or pharmacological treatment of viral particles. Any modification of the viral genome that attenuates or inactivates the virus can be determined by a test of infectivity in cell culture, where, using conventional techniques, the level of infectious viruses present in the culture supernatant is essentially reduced or eliminated compared to a wild type of virus.

The viral particle employed in this invention can be in isolated or purified form. The terms “isolated” or “purified”, as used in the context of this specification to define the purity of viral particles and compositions containing viral particles, means that the viral particles and compositions are substantially free of other proteins of natural or endogenous origin and contain less than about 1% by mass of protein contaminants residual of production processes. Such compositions, however, can contain other proteins added as stabilizers, excipients, or co-therapeutics. The viral particle is isolated if each viral protein contained in it is detectable as a single protein band in a polyacrylamide gel by silver staining.

In the preferred embodiments of the invention, it is the physical viral particle reconstituted in vitro that acts as an immunizing or vaccine agent eliciting a CTL response in a host or in an assay of the invention. This invention thus makes it possible to provide an immunizing or vaccinating viral particle without reconstitution of the viral particle after expression in a host cell in vivo. Viral particles can be employed in compositions that give rise to active agents capable of preventing the pathogenic effects of viral infection.

The evasiveness and diversity of viruses has made definitive treatment difficult. Presented here are methods and agents for preventing the spread of viral infections in a host, such as a human. Examples of enveloped viruses that can be employed in the invention are retroviruses, such as FeLV, FIV, HIV-1, HIV-2, SIV, MuLV, and GLV; herpes viruses, such as EBV, HSV, CMV, BHV-1, BHV-4, and pseudorabies virus; and paramyxovirus, such as Sendai virus, Newcastle disease virus, human parainfluenza 2 and 3, and mumps viruses, having fusogenic properties.

The term “fusogenic” is used herein in describing a property of the viral particles. Viral particles are fusogenic when they contain an envelope membrane, which, when the viral particles are targeted to professional antigen presenting cells (APCs) exhibit binding to the plasma membrane of the APCs and fuse with the plasma membrane of the APCs.

Binding of the viral particle to the receptor may involve noncovalent interactions between the viral particle envelope and the receptor, the sum of which leads to a high affinity, specific interaction between the viral particle and a cell surface molecule. Cell entry following fusion results in the virus crossing the plasma membrane and possible removal of the viral envelope. Following uptake of the viral particles by the APCs, the antigen of interest may be formed by degradation of proteins to produce peptides that combine with Class I MHC for exogenous antigen presentation.

As used herein, the term “exogenous antigen presentation” refers to antigen presentation following the uptake of an exogenous antigen with an APC by receptor-mediated binding and entry by surface fusion. This mode of antigen presentation is to be contrasted to endogenous antigen presentation in which an endogenous antigen is made within the presenting cell. Exogenous antigen presentation as referred to herein does not involve de novo synthesis of the antigen within the presenting cell (i.e., in situ).

As used herein, the term “professional antigen presenting cell” means a macrophage, dendritic, or B cell involved in the activation of antigen-specific niaeve T cells. These cells are adapted to present peptides and viral particles from different types of pathogens to T cells. The macrophage B cell and DC typically take up antigens by phagocytosis, endocytosis, macropinocytosis. Macropinocytosis refers to the uptake of a large volume of fluid or macrosolutes present in the extracellular milieu. Professional APC efficiently internalize specific antigens bound to their surface or present in the extracellular milieu.

If necessary, several experimental approaches can be employed to identify APCs that are fusogenic with the viral particles. For example, electron microscopy of newly infected APCs may demonstrate a majority of viral particles being internalized in endosomes or undergoing fusion at the cell surface. APCs that are not naturally fusogenic with the viral particles can be altered by gene transfer of receptor activity to normally receptor-negative cells or by stimulation of cells to enhance cell surface receptor concentration.

The viral particle is employed in the method of the invention in an effective amount sufficient to provide an adequate concentration of the drug to prevent or at least inhibit infection of the host in vivo or to prevent or at least inhibit the spread of the virus in vivo. Thus, the effective amount can be easily determined from the literature relating to the virus of interest.

The effective amount is preferably sufficient to induce protective immunity against the virus in a host to which the effective amount of the viral particle is administered. Thus, the protective immunity imparted by the method of the invention imparts, to an individual, protection from disease, particularly infectious disease associated with viral infection, as evidenced by the absence of clinical indications of disease, or as evidenced by absence of, or reduction in, determinants of pathogenicity, including the absence or reduction in persistence of the infectious virus in vivo, and/or the absence of pathogenesis and clinical disease, or diminished severity thereof, as compared to individuals not treated by the method of the invention.

The dosage of the viral particle administered to the host can be varied over wide limits. The viral particle can be administered in the minimum quantity, which is therapeutically effective, and the dosage can be increased as desired up the maximum dosage tolerated by the patient. The viral particle can be administered as a relatively high amount, followed by lower maintenance dose, or the parasite or viral mitogen can be administered in uniform dosages. The amount of the viral particles administered depends upon absorption, distribution, and clearance by the host. Of course, the effectiveness of the viral particles is dose related. The dosage of the viral particles should be sufficient to produce a minimal detectable effect, but the dosage should be less than the dose that activates a CTL response.

The dosage and the frequency of administration will vary with the viral particle employed in the method of the invention. Optimum amounts can be determined with a minimum of experimentation using conventional dose-response analytical techniques or by scaling up from studies based on animal models of disease.

The dose of the viral particle is specified in relation to an adult of average size. Thus, it will be understood that the dosage can be adjusted by 20-25% for patients with a lighter or heavier build. Similarly, the dosage for a child can be adjusted using well known dosage calculation formulas.

The term “about” as used herein in describing dosage ranges means an amount that is equivalent to the numerically stated amount as indicated by the induction of a CTL response in the host to which the viral particle is administered, with the absence or reduction in the host of determinants of pathogenicity, including an absence or reduction in persistence of the infectious or virus in vivo, and/or the absence of pathogenesis and clinical disease, or diminished severity thereof, as compared to individuals not treated by the method of the invention.

In practicing the treatment method of the invention, the viral particles can be administered to a host using one of the modes of administration commonly employed for administering drugs to humans and other animals. Thus, for example, the viral particles can be administered to the host by the oral route or parenterally, such as by intravenous or intramuscular injection. Other modes of administration can also be employed, such as intrasplenic, intradermal, and mucosal routes. For purposes of injection, the viral particles described above can be prepared in the form X of solutions, suspensions, or emulsions in vehicles conventionally employed for this purpose.

It will be understood that the viral particles can be used in combination with other prophylactic or therapeutic substances. For example, mixtures of different viral particles can be employed in the method of the invention. Similarly, mixtures of viral particles can be employed in the same composition. The viral particles can also be combined with other vaccinating agents for the corresponding disease, such as microbial immunodominant, immunopathological and immunoprotective epitope-based vaccines or inactivated attenuated, or subunit vaccines. The viral particles can even be employed as adjuvants for other immunogenic or vaccinating agents.

The viral particle can be used in therapy in the form of pills, tablets, lozenges, troches, capsules, suppositories, injectable in ingestable solutions, and the like in the treatment of cytopatic and pathological conditions in humans and susceptible non-human primates and other animals. Specifically, the host or patient can be an animal susceptible to infection by the virus, and is preferably a mammal. More preferably, the mammal is selected from the group consisting of a rodent, especially a mouse, a dog, a cat, a bovine, a pig, and a horse. In an especially preferred embodiment, the mammal is a human.

Appropriate pharmaceutically acceptable carriers, diluents, and adjuvants can be combined with the viral particles described herein in order to prepare the pharmaceutical compositions for use in the treatment of pathological conditions in animals. The pharmaceutical compositions of this invention contain the active viral particles together with a solid or liquid pharmaceutically acceptable nontoxic carrier. Such pharmaceutical carriers can be sterile liquids, such as water an oils, including those of petroleum, animal, vegetable, or synthetic origin. Examples of suitable liquids are peanut oil, soybean oil, mineral oil, sesame oil and the like. Water is a preferred carrier when the pharmaceutical composition is administered intravenously. Physiological solutions can also be employed as liquid carriers, particularly for injectable solutions.

The ability of the vaccines of the invention to induce protection in a host can be enhanced by emulsification with an adjuvant, incorporation in a liposome, coupling to a suitable carrier, or by combinations of these techniques. For example, the vaccines of the invention can be administered with a conventional adjuvant, such as aluminum phosphate and aluminum hydroxide gel. Similarly, the vaccines can be bound to lipid membranes or incorporated in lipid membranes to form liposomes. The use of nonpyrogenic lipids free of nucleic acids and other extraneous matter can be employed for this purpose.

Suitable pharmaceutical excipients include starch, glucose, lactose, sucrose, gelatine, malt, rice, flour, chalk, silica gel, magnesium carbonate, magnesium stearate, sodium stearate, glycerol monstearate, talc, sodium chloride, dried skim milk, glycerol, propylene glycol, water, ethanol, and the like. These compositions can take the form of solutions, suspensions, tablets, pills, capsules, powders, sustained-release formulations and the like. Suitable pharmaceutical carriers are described in “Remington's Pharmaceutical Sciences” by E. W. Martin. The pharmaceutical compositions contain an effective therapeutic amount of the viral particle together with a suitable amount of carrier so as to provide the form for proper administration to the host.

Another aspect of the invention includes administering nucleic acids encoding viral particles with or without carrier molecules to an individual. Those of skill in the art are cognizant of the concept, application, and effectiveness of nucleic acid vaccines (e.g., DNA vaccines) and nucleic acid vaccine technology as well as protein and polypeptide based technologies. The nucleic acid based technology allows the administration of nucleic acids encoding viral particles, naked or encapsulated, directly to tissues and cells without the need for production of encoded proteins prior to administration. The technology is based on the ability of these nucleic acids to be taken up by cells of the recipient organism and expressed to produce viral particles to which the recipient's immune system responds. Such nucleic acid vaccine technology includes, but is not limited to, delivery of naked DNA and RNA and delivery of expression vectors encoding the viral particles. Although the technology is termed “vaccine”, it is equally applicable to immunogenic compositions that do not result in a complete protective response. Such partial-protection-inducing compositions and methods are encompassed within the present invention.

Although it is within the present invention to deliver nucleic acids encoding the viral particles as naked nucleic acids, the present invention also encompasses delivery of nucleic acids as part of larger or more complex compositions. Included among these delivery systems are viruses, virus-like particles, or bacteria containing the nucleic acids encoding the viral particles. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical patents), which are incorporated by reference herein, and can be made and used without undue or excessive experimentation.

Whatever vaccine approach is used (genetic vaccination, pseudo-particles, inactivated or attenuated viruses, subunit vaccines, viral or non-viral vectors), the approach chosen should have the ability to enter effectively into the professional APC. This is particularly true when plasmid vectors (vaccination by DNA) or inactivated viruses are used. Likewise, immunotherapy protocols that are based on the ex vivo transduction of DC autologs prior to their being re-injected into a patient must take into account the efficacy of the phenomenon described herein. For example, using a defective HIV-1 virus for replication while retaining its fusogenic properties can offer various advantages. Unlike viral or lentiviral vectors that express interesting antigens, the lack of integration of viral material avoids potential problems caused by adding foreign genetic material into cellular DNA: the risk of transformation, mobilization of the viral genome integrated during infections, etc. Moreover, from an immunological perspective, the continuous expression of viral antigens can be harmful. Exogenous presentation by MHC-1 molecules will be a priori transient and ought not have this drawback. Relative to a subunit vaccine, moreover, an inactivated virus's ability to cover the whole viral epitope inventory is of interest.

Other applications of the exogenous presentation of HIV antigens can also be proposed. It is conceivable to inject not DNA but rather the viral particles produced in cell culture. One would thus be free of possible safety issues associated with the injection of DNA. One could use inactivated viruses (2, 37) or HIV lentiviral vector particles (32). In the latter case, the particles would be produced in the absence of the vector element so as to produce “empty” vectors. These same particles could also be used to pulse ex vivo some DC before re-injection.

The VSV-G envelope is certainly immunogenic on its own, but the VSV virus is not pathogenic in humans. Thus, a vaccination that is based on a vector expressing all or some of the proteins of HIV-1 is conceivable. It will be important to keep the envelope's fusogenic capabilities and to make the virus unable to replicate by mutation or by eliminating different genes (such as integrase, reverse transcriptase, etc.) and different sequences of the viral genome needed for reverse transcription and for integration.

The experimental system of the invention makes it possible to test the capacity of different vectors to enable the preparation of viral epitopes as well as their presentation by MHC-I molecules in the DC. Hence, this is very useful for analyzing a vaccine preparation's capability to stimulate specific CTL. The principle of the test is simple. DC or other professional APCs are exposed to virus and, after an incubation period, the capability of the DC or other professional APCs to activate the CD8+ effector cells is analyzed, such as the line EM71-1 described herein. Other effector lines or clones can also be used if the desire is to examine the presentation of other viral epitopes.

A preliminary experiment was performed using HIV virus inactivated for example, by Aldrithiol-2. This product inactivates the infectiveness of retroviruses by means of covalent bonding with the Zn fingers of NC and without affecting the envelope's fusogenic capabilities (2, 28, 29, 37). In a preliminary experiment, it was observed that inactivated viruses retain their ability to activate anti-HIV CTL. Other vectors, viral or not, can be analyzed by using the experimental system of this invention. An in vitro analysis of a vaccine preparation enables the selection of cells that would be worth testing in vivo as immunogenic agents or vaccines.

Plasmids containing the polynucleotides that encode viral particles and components thereof for use in the invention:

Gag expression vector (pR8.2, also called pCMV-R8-2 or pCMV-Gag in the text) described by the team of D. Trono (27).

Plasmid pCMV-VSV (47)

Inactivated virus (28-29); the virus can be inactivated by Aldrithiol or other ways

Viral particles (27)

HeLa cells (35)

Cell 293 (27)

This invention will be described in greater detail in the following Examples.

### EXAMPLE 1

MHC-I exogenous presentation of viral epitopes derived from incoming HIV-1 virions.

(A) Generation of Mononuclear Subsets.

DCs were prepared as described ⁴². Briefly, PBMCs were cultured 7 days in serum-free AIM-V medium (Gibco) containing 500 U/ml GM-CSF (a kind gift from Novartis) and 50 ng/ml IL-13 (Sanofi), and DCs were isolated by elutriation. Isolation procedure gave rise to CD1a+MHC-I+, MHC-II+, CD64−, CD83−, CD80 low, CD86 low cells, a phenotype corresponding to immature DCs. DC purity was >95%.

Monocyte-derived macrophages were obtained by adherence of PBMCs and cultured 7 days before use. Cells were >90% CD14+.

CD4+ T lymphocytes were obtained from PBMCs by negative selection with anti-CD8 magnetic beads (Dynal). Cells were activated by PHA and cultivated in the presence of 100 U/ml IL-2 (Chiron). Cells were 93% CD4+ CD3+.

HLA-A2 expression was determined by flow cytometry. C1R-A2 and C1R-B53 cells (a kind gift of F. Latron and M. Takigushi, respectively) and B-EBV-transformed cells were grown as described ⁴³.

(B) Viruses.

Replicative HIV-1 (X4-tropic HIV_{BRU} and HIV_{NL43}, and R5-tropic HIV_{JRCSF} strains) and env-deleted viruses pseudotyped with VSV-G or HIV-1 (from the X4-tropic HIV_{HXB2} strain) envelopes were produced by transfection as described ³⁵. Infectious titers, measured in single cycle assays using HeLa-CD4+CCR5+ reporter cells, were routinely around 500 and 5000 pfu/ng of p24, for viruses bearing an HIV-1 or a VSV envelope, respectively. When indicated, fusion-defective VSV-G (mutant Q117N) or HIV_{HXB2} (mutant F522Y, a kind gift of F. Mammano) envelopes ^{30,31} were used for pseudotyping. Viral infectivity of env-deleted or fusion defective virions was fully abrogated in single-cycle assays (not shown). Mutant envelopes are known to bind their receptors efficiently ^{30,31} and were normally incorporated into virions (not shown). When necessary, viral supernatants were concentrated and purified using 100-kDa-cutoff centrifugal concentrators (Millipore). HIV-vector (encoding for β-galactosidase) was prepared as described ²⁷. Infectious titer was around 4000 β-galactosidase units/ng of p24. The X4-tropic HIV_{MN} strain was prepared and inactivated by AT-2 as described ^{28,29} Infectious titers were 2×10⁶ and <1 pfu/ml, for non-treated and AT-2-inactivated HIV_{MN} viruses, respectively. The lack of in vitro infectivity of AT-2-inactivated virions has been confirmed by direct intravenous infusion of large amounts of inactivated SIV into macaques, without evidence of infection (Lifson et al, in preparation).

(C) CTL Lines.

The CTL line EM71-1 was derived from a child prenatally infected with HIV-1, by repeated stimulations of PBMC with irradiated autologous B-EBV cells coated with the p17 Gag peptide SLYNTVATL (SL9, originally described by 44) in the presence of allogeneic irradiated PBMCs. Peptide recognition was HLA-A2 restricted (SD50: 0.5 ng/ml in ⁵¹Cr assays). This epitope is present in HIV_{BRU}, HIV_{MN} and HIV_{JRCSF} strains and in HIV-vector.

In Elispot assays, production of IFN- was detected when C1R-A2 cells were pulsed with 0.01 ng/ml of peptide, and reached a plateau at 0.1 ng/ml. 97% of EM71-1 cells were CD8+, CD3+ and 94% SL9-HLA-A2 tetramers+ (a gift from F. Romagné, Beckman-Coulter-Immunotech). CTL culture conditions were as described ⁴³.

An HLA-A2+ CTL line (EM45) was derived from another HIV+ patient by stimulation with SL9 peptide. EM45 cells behave similarly as EM71-1 cells in HIV-1 virions cross-presentation assays (not shown). The CTL clone 141 (ref. ³²) recognized the p24 Gag epitope QASQEVKNW (QW9) in an HLA-B53 restricted manner (F.B., unpublished results). This epitope is present in HIV_{NL43} and in HIV-vector.

(D) CTL Assays.

AZT (5 μM, Sigma) was added to cells 3-5 hours before exposure to viruses and maintained throughout the assays. For Elispot assays, 2×10⁶ C1R-A2, T-lymphocytes or DCs (in 1 ml) were exposed to indicated viruses for 1 hour and diluted twice in fresh medium before overnight incubation. Macrophages (3-4×10⁶ cells per flask) were exposed to indicated viruses in 2 ml for 1 hour, in the presence of 1.5 ng/ml—CSF (R&D) and four-fold diluted before overnight incubation. Viral inoculum was 300 ng of p24/10⁶ CD4+ lymphocytes or C1R-A2 cells. In DCs, viral inoculum (in ng of p24/10⁶ cells) was 300 ng for HIV-vector and HIV strains, 500 ng for HIV(VSV) or HIV(HIV) pseudotypes and 1000 ng for untreated or AT-2-inactivated HIVMN virions. In macrophages, inoculum was 1000 ng of p24/10⁶ cells. As a positive control, stimulators were pulsed with the SL9 peptide (1 μg/ml). During assays, DCs were incubated in AIM-V medium containing IL-13 and GM-CSF and other cells in RPMI containing 10% FCS. Stimulator cells were washed twice before incubation with effectors. IFN-γ production by EM71-1 effector cells was measured in a Elispot assay adapted from ⁴⁵. Briefly, targets and effectors were incubated overnight in nitrocellulose-bottomed 96-well plates (Millipore) coated with anti-IFN-γ mAb 1-D1K (15 μg/ml, Mabtech). IFN-γ production was revealed by sequential incubations with biotinylated anti-IFN-γ mAb 7-B6-1 (1 μg/ml, Mabtech), streptavidine-alcaline phosphatase (0.5 U/ml, Boehringer Mannheim) and BCIP-NBT substrate (Promega). Positive spots were counted using a binocular microscope. For ⁵¹Cr release assays, 10⁶ target cells in 1 ml were exposed to the indicated virus (500 ng of p24) for 1 hour and diluted twice in fresh medium before overnight incubation, unless otherwise mentioned. ⁵¹Cr release assays were performed as described ⁴³.

(E) MHC-I Exogenous Presentation of Viral Epitopes Derived from Incoming HIV-1 Virions.

Whether primary immature DCs, macrophages, or CD4+lymphocytes process and present viral epitopes derived from incoming cell-free HIV-1 virions to CD8+ T lymphocytes was examined. Primary cells were prepared from HLA-A2+ HIV-seronegative individuals and exposed to HIV-1 virions as described above. The reverse transcriptase inhibitor AZT was added throughout the experiments to ensure that any presented antigens were derived from the input virus, and not from newly synthesized proteins.

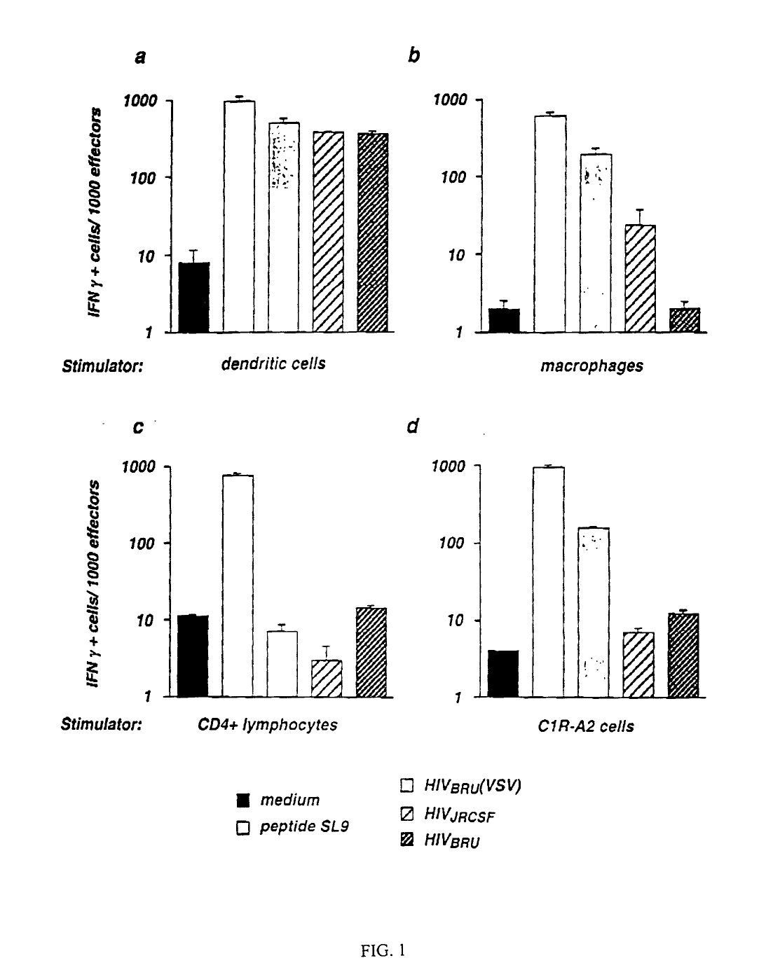

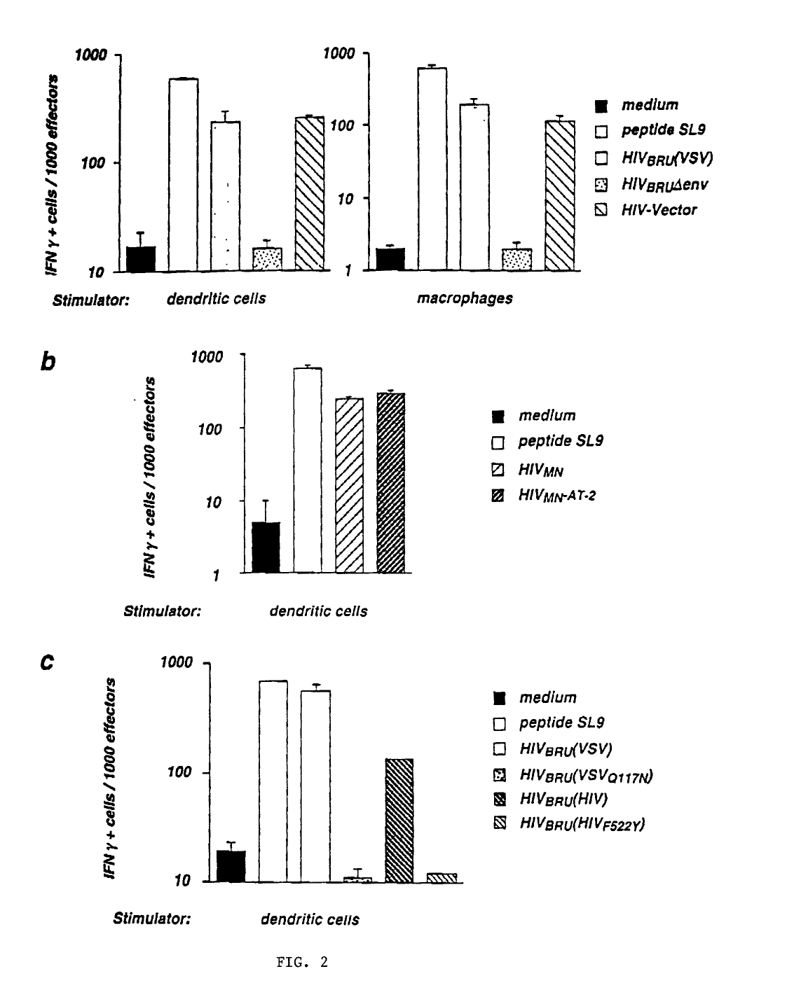

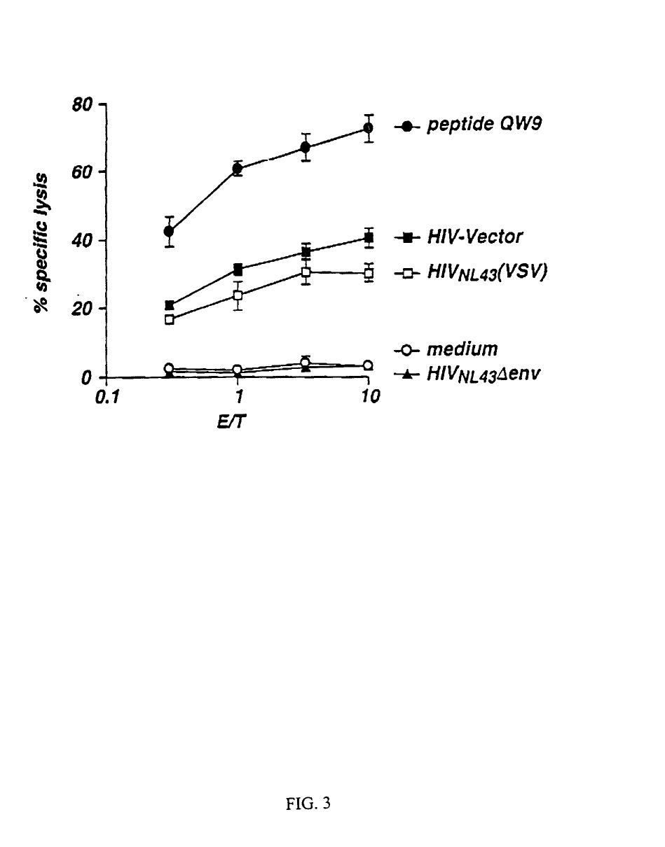

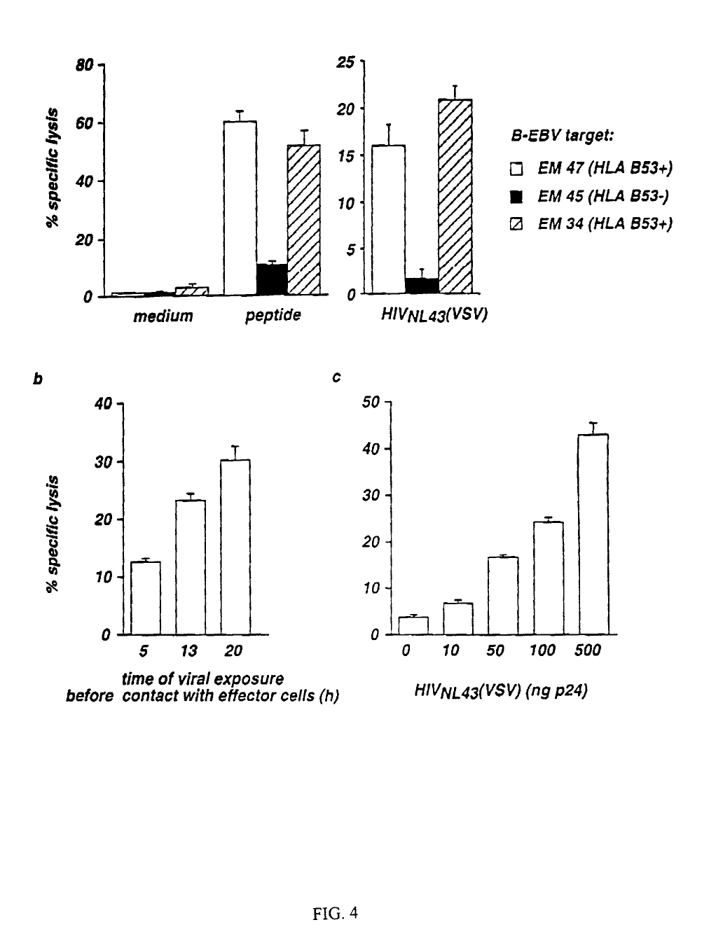

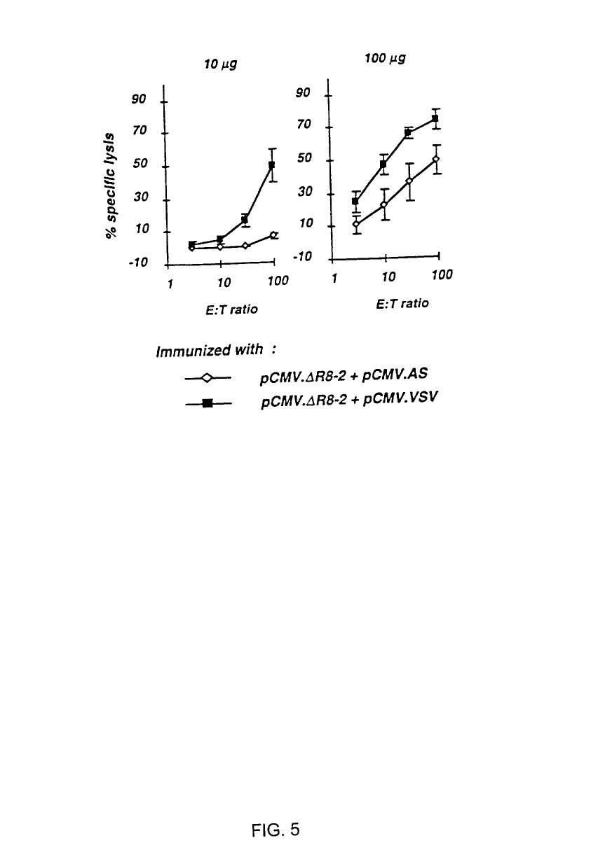

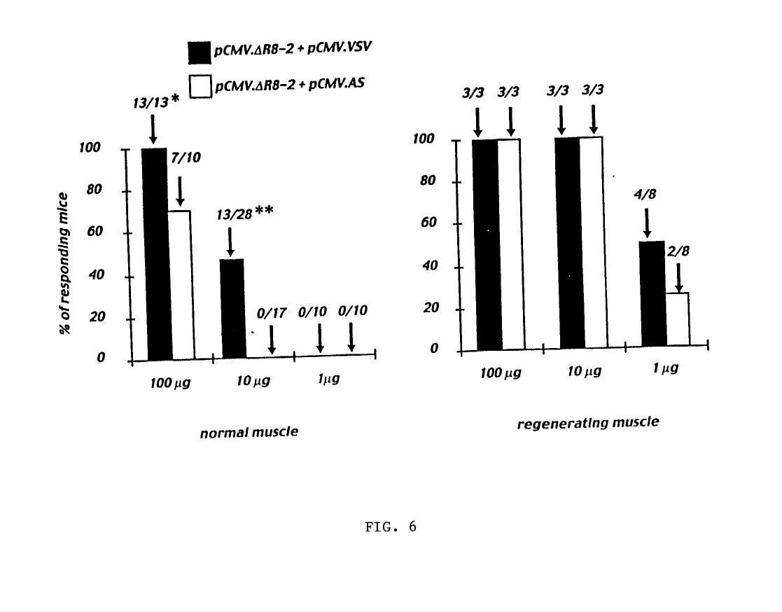

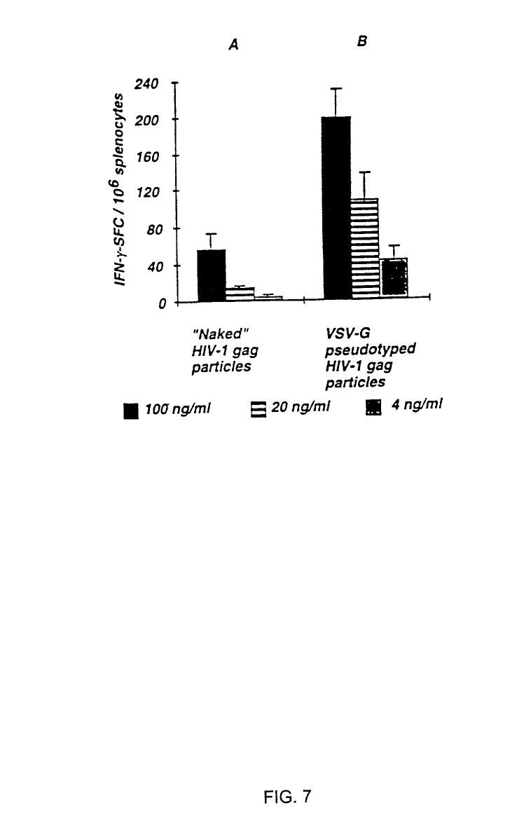

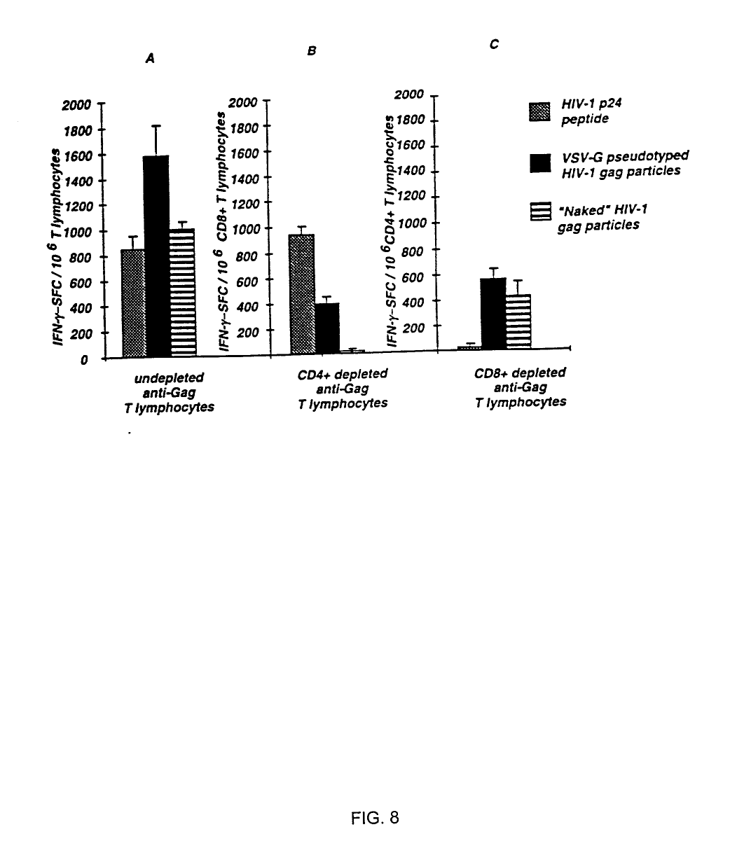

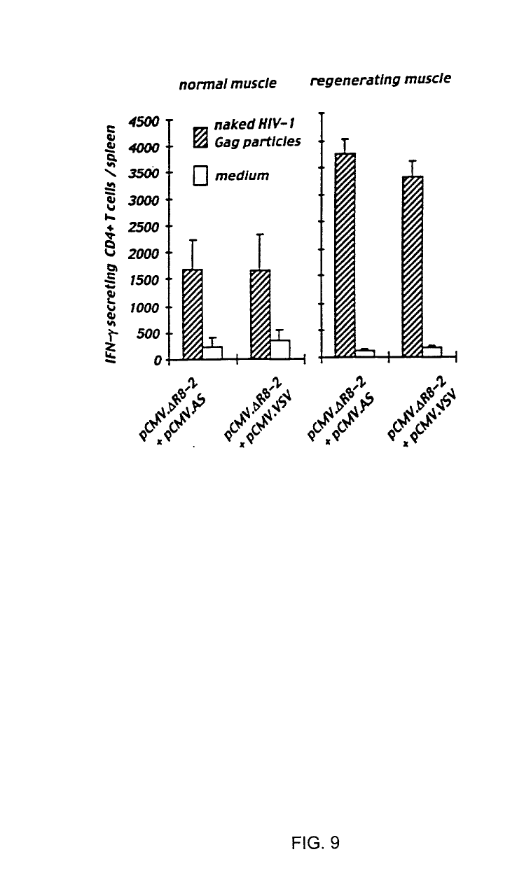

After viral exposure, cells were incubated with an HLA-A2-restricted CD8+ CTL cell line (EM71-1). The EM71-1 cells were derived from an HIV-infected patient and recognize a well-characterized immunodominant epitope of the Gag p17 protein. DCs, macrophages, or lymphocytes pulsed with the synthetic Gag peptide corresponding to optimal epitope (SL9) specifically activated EM71-1 cells, as measured by IFN-γ production (FIG. 1).

The ability of immature DCs to present exogenous HIV-1 antigens was first tested. AZT-treated DCs were exposed to the X4-tropic strain HIV_{BRU} or to the R5-tropic strain HIV_{JRCSF} and incubated with the EM71-1 cells. This resulted in a strong activation of EM71-1 cells. The results are shown in FIG. 1a.

HIV-1 particles can be pseudotyped with heterologous viral envelope proteins, such as vesicular stomatitis virus glycoprotein (VSV-G) ²⁵, resulting in the infection of a broad range of target cells. Whether DCs capture and process HIV virions pseudotyped with the VSV-G envelope (HIV_{BRU}(VSV)) was examined. The EM71-1 cells efficiently recognized DCs exposed to HIV_{BRU}(VSV) (FIG. 1a). Therefore, DCs present Gag epitopes upon exposure to incoming virions in the absence of viral protein neosynthesis. The exogenous presentation is observed with virions bearing either HIV-1 or VSV envelope glycoproteins.

Primary macrophages were then used as stimulators. AZT-treated HLA-A2+ macrophages activated EM71-1 cells after exposure to HIV_{JRCSF} virions and to HIV_{BRU}(VSV) pseudotypes (FIG. 1b). IFN-γ production by EM71-1 cells was, however, noticeably lower than when DCs were used as stimulators (compare FIGS. 1a and 1b). Macrophages, which presumably do not express a functional CXCR4 co-receptor ²⁴, did not stimulate EM71-1 cells upon exposure to HIV_{BRU} (FIG. 1b). Thus, macrophages present viral epitopes derived from incoming HIV-1 particles, albeit less efficiently than DCs.

Both X4- and R5-tropic HIV-1 strains actively enter and replicate in CD4+ lymphocytes. Whether exogenous presentation of HIV-1 antigens occurred in these cells was examined. HLA-A2+ CD4+ lymphocytes activated EM71-1 cells after pulsing with the cognate peptide (FIG. 1c). However, AZT-treated CD4+ lymphocytes exposed to HIV_{JRCSF} or HIV_{BRU}, as well as to HIV_{BRU}(VSV), failed to stimulate EM71-1 cells (FIG. 1c). Therefore, T lymphocytes, which are not professional APCs, did not present a Gag epitope derived from incoming virions. These results are in agreement with a previous observation that lysis of T lymphocytes by specific CTLs correlates with de novo synthesis of HIV proteins ²⁶. They also confirm that processing of exogenous antigens is restricted to professional APCs.

B cells are also APCs, but they do not express the HIV receptor CD4. To examine whether B cells process and present HIV-1 epitopes through the MHC-I-restricted exogenous pathway, the B cell line C1R expressing HLA-A2 (C1R-A2) was used as stimulator. AZT-treated C1R-A2 cells exposed to HIV_{BRU}(VSV) pseudotypes, but not to HIV_{JRCSF} nor HIV_{BRU}, stimulated EM71-1 cells (FIG. 1d). Thus, B lymphoid cells present epitopes from incoming virions coated with a VSV-G, and not with an HIV-1 envelope glycoprotein, probably because of the absence of the adequate viral receptors. Altogether, these results indicate that activation of specific CTLs by APCs, but not by CD4+ lymphocytes, can be achieved without neosynthesis of viral proteins. They also suggest that appropriate envelope-receptor interactions are required.

### EXAMPLE 2

(A) HIV-1 Gag and VSV-G Expression Vectors.

The HIV-1 Gag expression vector pCMV.ΔR8-2 is a kind gift of D. Trono (27). It drives the synthesis of all HIV-1 proteins besides Env. pCMV-VSV, a kind gift of A. Miyanohara, carries the VSV G glycoprotein (VSV-G) gene under the control of human cytomegalovirus immediate early promoter (47) [Yee, 1994 #2398]. pCMV.AS is a control plasmid carrying the VSV gene in the antisense orientation. It was constructed by inverting a BamHI-BamHI fragment encompassing the VSV-G gene in pCMV-VSV. HIV-1 Gag particles pseudotyped with the VSV G glycoprotein (HIV(VSV) particles) were produced by cotransfecting pCMVΔR8-2 and pCMV-VSV plasmids (at a 3:1 ratio) in HeLa cells as previously described (35). Naked HIV-1 Gag particles were produced by using PCMV.AS instead of pCMV-VSV. Stocks were analyzed for their HIV-1 p24 content by ELISA (Dupont de Nemours) and frozen.

(B) MHC-I Restricted Exogenous Presentation Occurs with Virions Incapable of Viral Protein Neosynthesis.

It was important to rule out the possibility that activation of effectors by APCs resulted from low levels of Gag protein, which could be synthesized despite AZT treatment. Two additional lines of experimental evidence confirmed that the activation was attributable to presentation of exogenous antigen. First, infectious HIV was replaced by an HIV-vector. HIV-vector particles consist of an HIV-1 capsid containing Gag and Pol-derived proteins and of a VSV-G envelope. The vector genome does not encode for any HIV-1 protein ²⁷. Exposure of DCs or macrophages to HIV-vector activated EM71-1 cells as efficiently as infectious HIV(VSV) pseudotypes (FIG. 2a).

Aldrithiol-2 (AT-2) inactivated HIV-1 virions ^{28,29} were then used. AT-2 covalently modifies the cysteines of the essential zinc fingers in the virion nucleocapsid protein, thereby fully inactivating viral infectivity. However, AT-2-inactivated virions retain the conformational and functional integrity of their gp120/gp41 complexes. AT-2-inactivated virions bind to and fuse with target cells, but the viral life cycle is arrested before initiation of reverse transcription ^{28,29}. Exposure of DCs to AT-2-inactivated HIV_{MN} activated EM71-1 effectors as efficiently as a matched preparation of infectious HIV_{MN} (FIG. 2b). Therefore, the use of HIV-vector particles and of AT-2-inactivated virions excluded a contribution of de novo viral protein synthesis during exogenous MHC-I presentation of HIV-1 antigens by DCs.

The possibility that effectors would be activated by free peptides or by soluble viral or cellular proteins was also eliminated. This was ensured by the purification of viral preparations with a 100-kDa-cutoff concentrator and by the fact that envelope-defective virions, expected to contain similar levels of contaminating soluble viral or cellular proteins, were ineffective (see below). The failure of CD4+ lymphocytes, which are efficient presenters of synthetic peptides, to present incoming HIV-1 virions (FIG. 1c) provides additional evidence that presentation is not due to peptide contamination of virus preparations. This shows that activation of effector cells by APCs was induced by epitopes derived from incoming virions and not by other sources of antigens.

### EXAMPLE 3

### Exogenous MHC-I Presentation of Epitopes Derived from Incoming Virions is Envelope-Dependent

The role of viral envelope glycoproteins in MHC-I-restricted presentation of HIV-1 Gag epitopes by APCs was studied. When exposed to virions devoid of viral envelope (HIV_{BRU} env), HLA-A2+ DCs and macrophages failed to activate EM71-1 effectors (FIG. 2a). These results, altogether with the observation that B cells exposed to virions bearing an HIV-1 envelope did not stimulate EM71-1 effectors (FIG. 1b), showed that exogenous presentation of a Gag epitope requires an interaction between the viral envelope protein and its receptors. To document this point further, virions coated with a fusion-defective HIV-1 envelope that retains the ability to bind CD4 (F522Y mutant) ³⁰ was used. DCs exposed to fusion-defective HIV-1 did not activate EM71-1 cells (FIG. 2c). Similar results were obtained with virions pseudotyped with a fusion-defective VSV-G protein (Q117N mutant) ³¹ (FIG. 2c). Thus, exogenous presentation of Gag epitopes requires a receptor-dependent fusion event. Requirement for membrane fusion indicates that the processing of HIV-1 antigen leading to cross-presentation necessitates the entry of viral proteins into the cytosol.

### EXAMPLE 4

### Exogenous Presentation Occurs with Various Viral Epitopes and MHC-I Molecules

An examination was made to determine whether exogenous presentation of HIV-1 antigens is observed with other viral epitopes and MHC-I molecules. To this aim, a second CD8+ effector cell line was used. The HLA-B53 restricted CTL clone 141 was derived from an HIV-infected patient. It recognizes an epitope from the HIV_{NL43} Gag p24 protein ³². B cells expressing HLA-B53 (C1R-B53 cells) were exposed to HIV_{NL43}(VSV) pseudotypes in the presence of AZT. A standard ⁵¹Cr release assay was performed 20 h later. CTL clone 141 efficiently killed C1R-B53 cells that had been exposed to HIV_{NL43}(VSV) (FIG. 3). Moreover, HIV-vector elicited killing activity of effectors as efficiently as infectious HIV_{NL43}(VSV), demonstrating that lysis of target cells was not due to de novo viral protein synthesis (FIG. 3). When exposed to virions devoid of viral envelope (HIV_{NL43} env), C1R-B53 cells were not killed by CTL clone 141 (FIG. 3), confirming the importance of viral envelope glycoproteins in cross-presentation of HIV antigens. Taken together, the results indicate that APCs exposed to incoming virions present exogenous epitopes derived from either Gag p24 or p17 proteins. APCs presenting such exogenously-derived antigens can induce both IFN-γ production and target cell killing by specific CTLs.

### EXAMPLE 5

### MHC-I Restriction, Kinetic and Dose-Response Analysis of HIV-1 Cross-Presentation

A further investigation was made of the presentation of HIV-1 epitopes derived from incoming virions. B lymphoid cells lacking HLA-B53 were not killed by CTL clone 141 upon exposure to HIV_{NL43}(VSV) (FIG. 4a). Similarly, HLA-A2-negative DCs or macrophages exposed to various HIV strains were not recognized by HLA-A2-restricted EM71-1 cells (not shown). Thus, the exogenous presentation of HIV-1 epitopes is appropriately MHC-I restricted. Moreover, kinetic analysis indicated that exogenous presentation is rapid. Target cells were recognized by clone 141 as early as 5 h post-exposure to HIV_{NL43}(VSV) (FIG. 4b).

A dose-response analysis of the viral inoculum was also performed. When C1R-B53 cells were exposed to increasing concentrations of HIV_{NL43}(VSV), killing by CTL clone 141 appeared dose-dependent. The lower effective viral input was 50 ng/ml (or 2 nM) of p24 (FIG. 4c). Similar results were obtained with the EM71-1 cell line. Recognition by EM71-1 of HLA-A2+ DCs and macrophages exposed to HIV_{BRU}(VSV) cells was significant at ˜50 and 500 ng/ml of p24, respectively (not shown). Therefore, the process requires a viral inoculum in the nanomolar range, which corresponds to an input of ˜500 virions per target cell. Given that the ratio of infectious to total particles is estimated to be lower than 1/1 000 (ref ³³), these results indicate that exogenous presentation is observed at low multiplicity of infection (m.o.i.). This low m.o.i. is likely to be attained in vivo, especially during early stages of infection, where a massive accumulation of HIV-1 within lymphoid tissues, as well as plasma viral loads up to 10⁷ virions/ml have been described ^{33,34}.

### EXAMPLE 6

### Application to the In Vivo Generation and to the Stimulation of Cytotoxic Lymphocytes

The first application tested involved using the VSV envelope to foster the entry of Gag particles, and thus exogenous presentation, in APC. The model chosen was vaccination by DNA in mice. Specifically, a study was made to determine whether the co-injection of a VSV expression plasmid in the presence of a Gag expression vector would enable the in vivo anti-Gag cytotoxic response (46) to increase. The hypothesis was that viral particles pseudotyped by the VSV envelope produced in situ would be internalized and processed by APC highly effectively. It could thus be hoped that the doses of plasmid needed to establish an immune response could be decreased. This parameter actually places a great restriction on DNA vaccination methodology when shifting from large animals to humans.

Techniques were used that were already laboratory-proven to induce an anti-HBV cytotoxic response (28). The Gag expression vector used (pR8.2) is the one described by the team of D. Trono (27). It codes for an HIV genome devoid of encapsulation sequences and the env gene, and it expresses the products of the gag, pot, nef, and tat genes under the control of a CMV promoter (27). It thus brings about the formation of Gag particles containing no viral genetic material.

The pCMV-VSV expression plasmid contains the gene of the VSV G envelope protein under the control of the CMV promoter (31). Also a plasmid was constructed containing the VSV G antisense gene (pCMV-AS) to assure that the possible effects observed would not be due to CpG type plasmid DNA sequences.

Female H-2^{d} BALB/c mice (iffa Credo, France) 6 to 8 weeks old, were used for immunogenicity studies. The HIV-1 Gag expression vector pCMV.ΔR8-2 was co-injected with either the VSV-G envelope encoding plasmid DNA (pCMV.VSV) or with a control plasmid carrying the VSV gene in antisense orientation (pCMVAS). DNAs were injected into normal or regenerating tibialis anteriro (TA) muscles as previously described (Mancini, J. Bio. Technics, 1996). Each TA received a total of 100, 10 or 1 μg of DNA composed with ¾ of pCMV.ΔR8-2 DNA and ¼ of pCMV.VSV or pCMV.AS DNA in a final volume of 100 μl. All intramuscular injections were carried out under anesthesia (sodium pentobarbital, 75 mg/kg, IP). All DNA vectors used for immunization were purified with Endofree Qiagen kits (Hilden, Germany).

The experimental protocol was as follows: intramuscular injection of a defined amount of plasmid into some BALB/c mice; the mice were killed 2 or 5 weeks later; splenocytes were put into culture in the presence of a synthetic peptide corresponding to a limited Gag H2d epitope; after one week of culturing, cytotoxic testing (release of radioactive chrome) using P815 cells as a screen (cells having the same haplotype as the mice), and pulsing with the synthetic peptide occurred. In an initial set of experiments, the muscle was pre-treated with cardiotoxin in order to bring about muscular regeneration, an inflammatory response, and an APC inflow (46). When the combination 75 μg pR8.2+25 μg pCMV-AS was injected, the appearance of anti-Gag CTL was observed in 2 mice out of 3 at 2 weeks post injection (see Table 1). In the presence of the VSV vector (combination of 75 μg pR8.2+25 μg pCM-VSV), the effectiveness seemed better since 3 mice out of 3 responded. The same results occur if P815 cells infected with a recombinant vaccine virus expressing the Gag protein are used rather than cells incubated with the peptide.

It was thus decided not to do a pretreatment with cardiotoxin and to vary the doses of plasmid injected (100 or 10 μg in total). With the 100 μg dose, a better CTL induction was observed in the presence of the VSV vector (16 responsive mice/16 mice treated with pCMV-VSV present, and 8/13 with no PCMV-VSV plasmid; see Table 1).

TABLE 1

| Amount of plasmid | | | |
|---|---|---|---|
| injected | Cardiotoxin | responsive mice/tested mice | |
| 100 μg | YES | PCMV GAG + | 2/3 |
| | | PCMV AS | |
| | | \overline{PCMV} GAG + | 3/3 |
| | | PCMV-VSV | |
| 100 μg | NO | PCMV GAG + | 8/13 |
| | | PCMV AS | |
| | | \overline{PCMV} GAG + | 16/16 |
| | | PCMV-VSV | |
| | | PCMV GAG + | 0/20 |
| | | PCMV AS | |
| 10 μg | NO | \overline{PCMV} GAG + | 6/31 |
| | | PCMV-VSV | |
| | | \overline{Paw s}eparated | 0/14 |
| | | PCMV GAG + | |
| | | PCMV-VSV | |

When pCMV-VSV and pCMV-R8-2 plasmids were injected into separate paw (10 microgram dose) no anti-gag response was detected (0 mouse out of 14 injected). The difference is more noticeable with the 10 μg dose of injected plasmid. No anti-Gag CTL was detected in the absence of pCMV-VSV plasmid (0 mice/20 injected). When the plasmid was present, 6 out of 31 mice responded (see table 1). This observation suggests that the VSV-G does not have an intrinsic adjuvant activity to stimulate the immune response.

This group of experiments shows that the presence of pCMV-VSV plasmid increases the efficacy of the cytotoxic response occurring with respect to the number of mice responding. This increase in efficacy probably happens owing to better entry into the APC cells of the viral material.

### EXAMPLE 7

Cytotoxic T lymphocytes (CTL) play a key role in the adaptative immune response by eliminating cells infected with intracellular pathogens or bearing tumor-related antigens. DNA-based vaccines are being evaluated as an attractive alternative to conventional protein vaccines as they can induce potent CTL responses. Strong cellular and/or humoral immune responses have been elicited by injection of DNA vaccines in a variety of species including human (67, 100, 112). In vivo priming of CTL by DNA injection predominantly occurs by antigen transfer from DNA-transfected cells to antigen presenting cells (APC) (60, 66). The injection of DNA into muscle results in the uptake of DNA not only by myocytes but by the neighboring cells as well. These non-lymphoid tissues express the plasmid-encoded protein. Although directly transfected dendritic cells have been isolated following intradermal biolistic immunization (58), transfected APCs probably play a minor role when the DNA is injected via the intramuscular route. After DNA-based immunization the strength of the immune response is dependent on the nature of the antigen expressed by non-lymphoid tissues and on its transfer to bone marrow-derived APC (60). APCs capture exogenous antigen through multiple pathways, which may influence the efficiency of antigen processing and presentation. It is well known that distinct antigen processing pathways leading to antigen presentation by two separate MHC classes (classe I or II) are required for endogenous and exogenous antigens to stimulate either CD8+ or CD4+ T cells (70). During the past few years, this dichotomous processing pathway has became more complex as it is now well demonstrated that exogenous antigens are processed for alternative MHC-I restricted antigen presentation to CD8⁺ T cells by APC (77, 97, 118). The stimulation of naive CTL by peptides derived from exogenous proteins has been referred to as cross-priming (51, 74).

Enhancement of MHC-restricted antigen presentation and vaccine-elicited CTL responses has been demonstrated in mice and in non-human primates by using cytokine administration (48, 79, 116), by triggering of costimulatory molecules (76, 78) and by inducing Fas-mediated apoptosis (57, 103). We recently demonstrated that HIV-1 Gag epitopes are presented by MHC class I molecules in the absence of viral protein synthesis in primary human dendritic cells and macrophages in vitro after uptake of HIV-1 virions (55). This exogenous presentation requires interaction between viral envelopes and their receptors as well as the fusion activity of the viral envelope. This was observed with virions bearing either HIV-1 or VSV envelope glycoproteins (VSV-G). Thus, a rational strategy would be to take advantage of the VSV-G envelope fusogenic activity and receptor mediated entry to increase antigen uptake in vivo after DNA-based immunization.

It was investigated whether pseudotyping of Gag particles by the VSV-G envelope could enhance in vivo the Gag-specific immune response after DNA-based immunization. The results show that injection of plasmids encoding VSV-G-coated HIV-1 Gag particles improved the Gag-specific CD8+ T cell response in mice. This was confirmed by in vitro experiments indicating that VSV-G pseudotyping of Gag particles allowed the Gag protein to enter into the MHC class I pathway. Finally, this invention shows that both CD4+ and CD8+ T cell responses were improved after local recruitment of APCs, confirming the predominant role of these cells in uptake of the released antigen and immune response induction.

Materials and Methods

HIV-1 Gag and VSV-G Expression Vectors.

The HIV-1 Gag expression vector pCMV.ΔR8-2 is a kind gift of D.Trono (92, 119). It drives the synthesis of all HIV-1 proteins besides Env. The plasmid pCMV-VSV, a kind gift of A. Miyanohara, carries the VSV G glycoprotein (VSV-G) gene under the control of human cytomegalovirus immediate early gene promoter (117). pCMV.AS is a control plasmid carrying the VSV gene in anti-sense orientation. It was constructed by inverting a BamHI-BamHI fragment encompassing the VSV-G gene in pCMV-VSV. Plasmid pCMV-VSV mut encodes a fusion defective VSV-G protein (mutant Q117N) (114).

HIV-1 Gag particles pseudotyped with the VSV-G glycoprotein were produced by cotransfecting pCMV.ΔR8-2 and pCMV-VSV plasmids (at a 3:1 ratio) in HeLa cells as previously described (89). Naked HIV-1 Gag particles were produced by using PCMV.AS instead of PCMV-VSV. Stocks of purified particles were obtained after concentration of supernatants from transfected Hela cells using membranes with a cut-off value of 100 Kd. Quantification of the particles was done according to their HIV-1 p24 content by ELISA (Dupont de Nemours, France) and kept frozen at −70° C. before use.

DNA-Based Immunization.

Female H-2^{d} BALB/c mice (Iffa Credo, France) 6 to 8 weeks old, were used for immunogenicity studies. The HIV-1 Gag expression vector pCMV.ΔR8-2 was co-injected with either the VSV-G envelope encoding plasmid DNA (pCMV.VSV) or with a control plasmid carrying the VSV gene in antisense orientation (pCMV.AS). DNAs were injected into normal or regenerating (i.e. cardiotoxine treated) tibialis anterior (TA) muscles as previously described (87). Each TA received a total of 100, 10 or 1 μg of DNA composed of ¾ of pCMV.ΔR8-2 DNA and ¼ of pCMV.VSV or pCMV.AS DNA in a final volume of 100 μl. All intramuscular injections were carried out under anesthesia (sodium pentobarbital, 75 mg/kg, i.p.). All DNA vectors used for immunization were purified with Endofree Qiagen kits (Hilden, Germany).

CTL activity Assay.

Immunized mice were sacrificed and spleens were removed 2 weeks after DNA-based immunization. Splenocytes were cultured (10⁷ cells/well in 24-well plate) in 2 ml of a Minimum Essential Medium (α-MEM, Gibco, Cergy Pontoise, France) supplemented with 10 mM Hepes, non essential amino acids, 1 mM sodium pyruvate, antibiotics, glutamine (Gibco BRL, Cergy Pontoise, France), 0.05 mM β-mercaptoethanol, and 10% fetal calf serum (Myoclone, Gibco BRL). Splenocytes were stimulated with 1 μg/ml of HIV-1 p24 (Gag 62-76) peptide (GHQAAMQMLKETINEE) containing a H2d-restricted epitope (107). Five days later half of the medium was replaced with fresh medium and two days later cells were used as effectors for the measurement of specific cytolytic activity in a standard chromium release assay. The targets cells were H-²d murine mastocytoma cells (P815) pulsed with the HIV-1 p24 (Gag) H-2^{d} restricted peptide (15 μg/ml), or P815 cells infected with a recombinant vaccinia virus encoding the HIV-1 Gag protein (rvv TG 1144, (96) at a multiplicity of infection (MOI) of 20/1. Unpulsed P815 cells or wild type vaccinia virus infected cells were used as control. Targets were labeled with ⁵¹Cr (3.7 MBq/10⁶ cells, Amersham, U.K.). After a 4 h incubation at 37° C., 50 μl of supernatants were collected and counted on a beta counter as described (54). Spontaneous and maximum releases were determined from targets incubated with either medium alone or lysis buffer (5% Triton X-100, 1% SDS). Percentage of specific release was calculated as (experimental release—spontaneous release)/(maximum release—spontaneous release)×100. The specific lysis was determined for each point in triplicate.

ELISPOT Assay.

IFN-γ releasing cells were quantified after peptide or Gag particle stimulation by cytokine-specific enzyme-linked immunospot assay (ELISPOT). Flat-bottomed nitrocellulose ELISA plates (Multiscreen, Millipore, Molsheim, France) were coated with 50 μl of rat anti-mouse IFN-γ (5 μg/ml, Pharmingen, San Diego, Calif.) overnight at 4° C., and thereafter saturated for 2 hours at 37° C. with RPMI 1640 containing 10% of FCS. Splenocytes (1×10⁶/well in 96 well plates) were incubated 40 hours in complete a-MEM (see CTL activity) at 37° C. in 5% CO₂ using different antigenic stimulations. Cells were incubated with HIV-1 Gag peptide (1 μg/ml), with VSV-G pseudotyped HIV-1 Gag particles (100 ng/ml) or with “naked” HIV-1 Gag particles (100 ng/ml). Background was evaluated with cells in medium or in concentrated supernatants from untransfected Hela cells. Cells were removed by flicking the plates, then lysed with water. After washing with PBS 0.05% tween 20, biotinylated rat anti-mouse IFN-γ antibody (1μg/ml, Pharmingen, San Diego, Calif.) was added for 90 minutes incubation at room temperature. Wells were washed as above prior to incubation with streptavidin-alkaline phosphatase-conjugate (Boehringer-Mannheim, Germany) at 1:1000 dilution in PBS for 1 h 30 min. Then, a 2.3 mM solution of 5-bromo-4-chloro-3-indolyl phosphate BCIP and nitroblue tetrazolium NBT (Promega, Madison, Wis.) diluted in alkaline buffer solution was added. When spots were visible, the reaction was stopped with water and air-dried. The number of IFN-γ secreting blue spots was counted and results were expressed as single spot forming cells (SFC). Each cell population was titrated in triplicate, spots were counted double blind.

The percentage of CD8⁺ and CD4⁺ T cells was determined by FACS analysis of fresh splenocytes using direct staining with anti-mouse CD8⁺ FITC and CD4⁺ PE antibodies (Pharmingen, San Diego, Calif.). Depletion of CD8⁺ and CD4⁺ T cells from mouse splenocytes was achieved by magnetic cell sorting (MACS, Miltenyi Biotec, Paris, France) as previously described (88). The percentage of undesired cells in the depleted fraction was less than 0.4%.

Statistical Analysis.

Categorical variables were compared with the χ² Pearson test. The minimal p value for rejection of the null hypothesis, i.e. no difference between VSV-immunized and control group, was 0.05.

Co-injection of a Vector Coding for HIV-1 Gag Particles with a Plasmid Encoding the VSV-G Envelope Increases Gag-Specific Cytotoxic Responses In Vivo.

To investigate the role of VSV-G envelope pseudotyping in the in vivo uptake of Gag particles by APC, mice were immunized with a vector encoding Gag particles and a plasmid encoding or not the VSV-G envelope. Optimal results for the intracellular expression of antigens and the production of fusogenic HIV/VSV particles were obtained in vitro following co-transfection of Gag and VSV-G expression vectors in Hela cells (89). Immunofluorescence and confocal microscopy analysis performed on transfected cells indicated that both Gag and VSV-G antigens partially co-localized within the same cell (data not shown). Quantification of Gag-p24 in cell culture supernatant allowed us to choose a 3 to 1 DNA ratio of pCMV.ΔR8-2 and pCMV.VSV for in vivo injections. As control for pCMV.VSV injection, we used a vector containing the VSV-G coding domain in anti-sense orientation (pCMV.AS). The efficiency of co-injection of plasmids encoding “naked” Gag particles (pCMV.ΔR8-2+pCMV.AS) or of plasmids coding for VSV-G envelope-pseudotyped Gag particles (pCMV.ΔR8-2+PCMV.VSV) at inducing Gag-specific CTL in vivo was tested in mice. Groups of 5 B

ALB/c mice were injected once with 10 μg or 100 μg of total DNA i.m. into normal muscle. Cytotoxic CD8+ T cell response was tested 2 weeks later using splenocytes from immunized mice as effector cells and P815 cells pulsed with a MHC-class I-restricted Gag peptide or unpulsed cells as targets.

No specific lysis was observed for spleen cells derived from any of the five mice injected with 10 μg of plasmids encoding “naked” Gag particles. In contrast, cytotoxic T cells were found in the spleen five out of five mice immunized with 10 μg of vectors coding for VSV-G -pseudotyped Gag particles (FIG. 5, left). In addition, the Gag-specific cytotoxic activity of spleen T cells derived from mice immunized with 100 μg of pCMV.ΔR8-2+pCMV.VSV was significantly higher than that detected after immunization with pCMV.ΔR8-2+pCMV.AS (FIG. 5, right). The number of effector cells required for a 50% lysis of target cells was ten times lower for mice immunized with 100 μg of pCMV.ΔR8-2+pCMV.VSV DNA than for mice immunized with 100 μg of pCMV.ΔR8-2+PCMV.AS DNA (FIG. 5, right). The anti-Gag CTL response was also tested against P815 cells infected at a MOI of 20/1 with recombinant vaccinia virus encoding the HIV-1 Gag protein. Similar results were obtained (data not shown), confirming a more potent cytotoxic T cell response for mice receiving plasmids encoding VSV-G-pseudotypes than for mice injected with vectors coding for “naked” Gag particles. In addition, these results indicate that CTL induced after pCMV. ΔR8-2 injection recognized peptides derived from endogenously processed protein. These results show that co-injection of the plasmid encoding the VSV-G envelope significantly increases the magnitude of Gag-specific CTL response after pCMV.ΔR8-2 injection in vivo. This also indicates that Gag epitopes were better presented in vivo by the APC when Gag particles were pseudotyped with VSV-G envelope.

Dose-Dependent Gag-Specific Cytotoxic T Cell Responses after DNA-Based Immunization in Mice.

To evaluate whether the enhanced efficiency of Gag-specific immune response after co-injection of pCMV.VSV with pCMV.ΔR8-2 DNA would permit decreasing the dose of injected DNA, 1, 10, or 100 μg of DNA were injected into normal or regenerating muscle. The Gag-specific cytotoxic responses against P815 target cells pulsed with Gag peptide or infected with a recombinant vaccinia virus expressing HIV-1 Gag protein were evaluated 2 weeks after DNA injection as described above.

After co-injection into normal muscle of low doses of DNA plasmids (1 μg) encoding either the Gag particle alone (pCMV.ΔR8-2+pCMV.AS) or the Gag particle pseudotyped with the VSV-G envelope (PCMV.ΔR8-2+pCMV.VSV), no specific lytic activity against P815 cells pulsed with the Gag peptide was detected (see FIG. 6, left). At the dose of 10 μg of DNA, a significant number of mice with Gag-specific response (13/28, p<0.001) was observed following immunization with plasmids encoding the VSV-G-pseudotyped Gag particles. In contrast, none of the animals (0/17) injected with plasmids encoding the “naked” Gag particles responded at this dose. This result was further confirmed after immunization with 100 μg of DNA since a significantly higher number of mice (p<0.05) display a cytotoxic response after co-injection of plasmids encoding the Gag protein and VSV-G envelope (13/13 compared to 7/10, see FIG. 6, left).

It was previously shown that cardiotoxin allows destruction of muscles fibers followed by their regeneration. This results in a ten fold more efficient gene transfer in regenerating than in normal muscle (62). Furthermore the local inflammation leads to a better recruitment of antigen presenting cells (APC) to the site of injection, thus improving the immune response induced after DNA injection (85). The number of responding mice following injection in cardiotoxin-pretreated muscles of either 10 or 100 μg of vectors coding for “naked” or VSV-G-pseudotyped Gag particles was comparable and reached 100%. However, injection of 1 μg of DNA was sufficient to induce a specific CTL in the spleen from 4/8 mice immunized with plasmids encoding the Gag protein and the VSV-G envelope and in 2/8 mice immunized with plasmids encoding the Gag protein alone (FIG. 6 right panel).

These results indicate that under normal conditions, when the number of APC present at the site of DNA injection is low, pCMV.ΔR8-2 is more immunogenic when coinjected with a vector encoding the VSV envelope. Thus, in normal muscle, the use of VSV-G envelope to pseudotype Gag particles could reduce the amount of injected DNA.

It was also confirmed that recruitment of APC to the injection site strongly increases the number of mice displaying Gag-specific cytotoxic activity after DNA-based immunization.

Role of the VSV-G Envelope in Enhancement of the Cytotoxic Response.

In order to determine if the increase in cytotoxic activity (FIG. 5) and in the frequency of responding mice (FIG. 6) observed after co-immunization of pCMV.ΔR8-2 with pCMV.VSV result from the fusogenic property of the VSV envelope or from a possible adjuvant effect of the VSV-G protein per se, cytotoxic T cell responses were analyzed 2 weeks after injection of a total amount of 10 μg of DNA into normal muscle (Table 2).

TABLE 2
Adjuvant effects of VSV-G on Gag-specific CRL response

| | Number of | |
|---|---|---|
| injected DNA (10 μg)^{a} | responding mice^{b} | X² Pearson test |
| pCMV ΔR8-2 + pcmV.VSV | 13/28 (46%) | p < 0.001 |
| (same paws) | | |
| pCMV ΔR8-2 + pcmV.AS | 0/17 (0%) | |
| (same paws) | | |
| pCMV ΔR8-2 + pcmV.VSV | 13/28 (46%) | p < 0.05 |
| (same paws) | | |
| pCMV ΔR8-2 + pcmV.VSV mut | 2/14 (14%) | |
| (same paws) | | |
| pCMV ΔR8-2 + pcmV.VSV | 3/14 (21%) | p < 0.05 |
| (separate paws) | | |
| pCMV ΔR8-2 + pcmV.VSV | 0/17 (0%) | |
| (same paws) | | |

^{a}DNA was injected in normal muscle.
^{b}Splenocyte cytotoxic activity was measured against P815 target cells pulsed or not with HIV-1 Gag peptide.

First, DNA expressing Gag particles was coinjected with a plasmid encoding a VSV-G envelope devoid of fusogenic activity (114). A significantly decreased number of responding mice (2/14, 14%) was observed (p<0.05, Table 2) compared to what was obtained following coinjection in the same leg with plasmid encoding the fusogenic VSV envelope (13/28, 46%). This indicates that the fusogenic activity of the VSV-G envelope protein was necessary to the observed enhancement in cytotoxic responses in vivo. Next, to see whether the VSV-G protein could have an adjuvant effect per se, we injected the two plasmids were injected into separate legs. Co-injection of pCMV-VSV with Gag-expressing DNA at different sites gave 21% response rate (3/14 responding mice) compared to co-injection with control plasmid at the same site (0/17 responding mice). This indicated that VSV-G had an additional adjuvant effect on the Gag-specific immune response (p<0.05).

Altogether these results suggest that the observed increase in Gag-specific responses after pCMV-VSV co-injection was due in part to the fusogenic activity of the VSV-G envelope that allows an improved uptake and processing of the Gag particles by APCs as well as to the intrinsic immunological properties of VSV.

Pseudotyping of HIV-1 Gag-Particles with VSV-G Enhances the Presentation of HIV-1 Gag Epitopes In Vitro.

In order to get further insights in the mechanisms involved in the increased efficiency of DNA vectors encoding VSV-pseudotyped Gag particles for the induction of gag-specific cytotoxic responses in vivo, we studied the involvement of the VSV-G envelope in the in vitro uptake and processing of HIV-1 Gag particles was studied. Different concentrations of viral particles were tested for their ability to generate Gag epitopes after in vitro processing. As readout for the detection of epitopes derived from the processing of either naked or VSV-G-pseudotyped Gag particles, we used Gag-specific effector T cells, that were obtained from mouse spleen taken 2 weeks after injection into regenerating muscle of 100 μg of DNA vector encoding the Gag protein only (pCMV.ΔR8-2). These spleen cells contained macrophages, dendritic cells and B cells that could serve as APC for the processing of Gag particles and the presentation of Gag peptides to T cells. The number of epitope-specific T cells producing IFN-γ was measured in response to a short-term stimulation (40 h) of the splenocytes with either naked or VSV-G pseudotyped Gag particles. The number of Gag-specific IFN-γ producing T cells increased with the concentration of viral particles within the dose-range studied (FIG. 7). Interestingly, the number of IFN-γ spot-forming cells (SFC) was significantly higher when Gag particles were pseudotyped with VSV-G envelope (compare FIGS. 7A and 7B). This result shows that the presentation of Gag epitopes derived from the in vitro processing of exogenous Gag particles is much more efficient when viral particles are pseudotyped with a heterologous viral envelope such as VSV-G, than when they are in a naked form.

VSV-G-Pseudotyped Gag Particles Enter MHC Class I and Class II Pathways.

To determine if epitopes presented after in vitro processing of either “naked” or VSV-G-pseudotyped Gag particles were derived from the class I or the class II processing pathway, we simultaneously performed ELISPOT assay on undepleted (FIG. 8A), CD4⁺ T cell-depleted (FIG. 8B) and CD8⁺ T cell-depleted (FIG. 8C) splenocytes taken from mice immunized with the DNA vector encoding the Gag protein only.

Stimulation of undepleted Gag-primed spleen cells with VSV-G-pseudotyped Gag particle increased the number of IFN-γ secreting T cells as compared to stimulation with “naked” Gag particles (FIG. 8A). This confirms the more efficient presentation of Gag epitopes after in vitro uptake of VSV-pseudotyped particles by APC (see FIG. 7).

The number of specific T cells producing IFN-γ after stimulation with “naked” HIV-1 Gag particles was reduced to basal level following CD4⁺ T cell depletion (FIG. 8B). This indicates that epitopes derived from in vitro processing of “naked” Gag particles were recognized by CD4⁺ T cells only. In contrast, after either CD4⁺ T (FIG. 8B) or CD8⁺ T cell depletion (FIG. 8C), the number of specific T cells producing IFN-γ following stimulation with VSV-G pseudotyped Gag particles was decreased compared to undepleted splenocytes (FIG. 8A), but was not significantly different between CD4+ or CD8+ depleted splenocytes (compare FIG. 8B and FIG. 8C). This indicates that not only CD4⁺ T lymphocytes but also CD8⁺ T cells secreted IFN-γ after recognition of Gag epitopes presented on APC pulsed with VSV-G-pseudotyped particles.

Stimulation of undepleted primed spleen cells with the HIV-1-p24 Gag peptide also resulted in the production of IFN-γ secreting T cells. This suggests that part of the spots detected after stimulation with pseudotyped particles were due to the recognition of this epitope by specific T cells derived from pCMV.ΔR8-2-injected mice (FIG. 8A). The number of SFC stimulated with this peptide was decreased to basal level in a CD8⁺ T cell-depleted population (FIG. 8C) indicating that secretion of IFN-γ was due to recognition of the HIV-1 Gag epitope/MHC class-I complex by CD8⁺ T cells. In contrast, after depletion of CD4⁺ T lymphocytes (FIG. 8B) the number of peptide-specific T cells producing IFN-γ was not significantly different, indicating that this 16 amino acid Gag peptide was not recognized by CD4⁺ T cells obtained from pCMV.ΔR8-2 Gag-immunized mice.

All together these results suggest that, when Gag particles are pseudotyped with VSV-G envelope, the viral proteins protein enter both the MHC class I and class 11 processing pathways, whereas in the absence of VSV-G envelope, Gag particles gain access to the MHC class II processing pathway only.

Gag-Specific CD4⁺ T Cell Response In Vivo is not Dependent on the Presence of the VSV-G Envelope.

To confirm that the VSV-G pseudotyping of Gag particles had no effect on the Gag-specific CD4⁺ T cell response, we immunized mice with vectors encoding either “naked” or VSV-pseudotyped Gag particles. Spleens were taken from mice two weeks after a single injection of 100 μg of DNA into normal or regenerating muscles. CD4⁺ T cell response was quantified by an IFN-γ ELISPOT assay after a 40 h stimulation with “naked” gag particles that we have previously shown to be processed through the class II pathway only (see above). The frequency of Gag-specific CD4+ T cells producing IFN-γ was not significantly different in mice immunized with vectors coding or not for the VSV-G envelope (FIG. 9). The total number of specific CD4⁺ T cells per spleen was not different between these two groups either, but was two times higher in mice immunized following cardiotoxin pretreatment (FIG. 9). This indicates that pseudotyping Gag particles with the VSV-G envelope has no major effect on the generation of Gag-specific class II-restricted responses in vivo and underlines the importance of APC recruitment at the injection site for the induction of strong specific T cell responses.

The present invention shows that induction of HIV-1 Gag specific cytotoxic T cells can be increased in mice using VSV-G pseudotyped Gag particles administered by DNA-immunization. This operates through an improved receptor-mediated uptake and processing of the Gag particles by APC after fusion with the VSV-G envelope but also through intrinsic adjuvant properties of VSV-G protein. In contrast, the efficiency of the class II processing and presentation remained unchanged whether Gag particles were pseudotyped or not.

DNA-based immunization represents an efficient strategy to induce CTL in vivo. Direct injection of a plasmid DNA expression vector into skeletal muscles results in the synthesis of plasmid-encoded antigens in the host cells (61, 115). These foreign proteins are then subjected to natural immune surveillance by dendritic cells, resulting in both MHC class I and II cellular responses. Studies using bone marrow chimeras showed that antigenic peptides involved in priming a CTL response are presented in the context of MHC class I molecules on bone marrow-derived cells and not by myocytes (59, 65, 69). Thus, immune responses are initiated by antigen expressed by transfected dendritic cells (direct priming) or by nonlymphoid cells (cross-priming). However, depending on the nature of the antigen and its localization in the transfected cells immune responses could vary greatly (81, 84).

The VSV-G envelope allows HIV-1 entry through a pH dependent endocytic pathway (46). The chimeric viruses composed of HIV-1 core and the VSV-G envelope termed HIV-1 (VSV) pseudotypes have been shown to be much more infectious than non-pseudotyped HIV-1 virions due to the infection of a broad range of target cells through a fusion-dependent mechanism (92). It has been reported that non-replicating VSV/HIV-1 virus efficiently transduced DC at immature stage leading to further maturation and to efficient antigen presentation to CD4+ and CD8+ T cells from HIV-1 infected individuals (71). In addition, it has recently been shown that human dendritic cells can present Gag epitopes upon exposure to incoming virions bearing either HIV-1 or VSV envelope glycoproteins, and that this occurred in the absence of viral protein synthesis. However, a broader range of APCs were targeted when incoming virions were coated with VSV-G rather than with HIV-1 envelope (55).

To increase uptake efficiency of antigen produced in vivo after DNA-based immunization, we used the VSV-G envelope to pseudotype Gag particles. Our study was based on co-immunization of mice with DNA plasmids encoding either HIV-1 Gag particles only or Gag particles pseudotyped with the VSV-G envelope. We showed that, in vivo, the anti-Gag specific CTL response was strongly increased after co-injection with the VSV-G encoding plasmid. The number of mice with Gag-specific CTLs in spleen as well as the intensity of the cytotoxic response was significantly increased for two different doses of DNA in mice injected with plasmids allowing the formation of VSV-G-pseudotyped particles. In contrast, coinjection of mice with a vector coding for a VSV envelope devoid of fusogenic activity significantly reduced the number of mice with Gag-specific CTL. Injection of the DNA coding for Gag and for VSV-G at different sites led to a two-fold reduction of the number of mice with Gag-specific CTL. However, compared with mice receiving the pCMV.AS anti-sense vector, the number of responder mice was still significant. This suggests that production of VSV-G protein at a distant site induced an activation of the immune system that resulted, in turn, in an improvement of the Gag-specific CTL response. Indeed, it was found that injection of a plasmid encoding VSV-G induced a high frequency of VSV-specific IFN-γ-secreting CD4+ T cells (data not shown). Recently, the requirement of CD4+ T cell help for CTL priming was shown to act via cross-priming mechanisms involving APCs (49, 98, 105). This CD4+ T cell help was originally described to be antigen specific; however, a nonspecific stimulus through CD40 was shown to restore APC conditioning leading to CTL priming in MHC class II^{-/-} mice (49, 105). Recent data indicate that dendritic cells in plasmid DNA-injected mice require conditioning signals from MHC class II-restricted T cells that are both CD40-dependent and independent. The signals required for priming CTL from plasmid injection may be antigen-independent or nonspecific and provided by cytokine secretion (56, 86, 111). Thus, it is conceivable that VSV-specific immune response provide a non-specific T cell help for the generation of Gag-specific CD8+ T cell responses.

Additionally, VSV-G could exert a positive effect on particle infectivity by various ways. VSV-G-carrying vesicles are produced and efficiently released into culture medium from cells expressing VSV-G in the absence of other viral component (94). VSV-G could thus increase the release of Gag particles when VSV-G and Gag proteins are co-expressed in the same cell. Moreover, it has been reported that VSV-G can be incorporated in naked HIV-1 particles after virion release (108), providing another mechanism for increasing viral infectivity. It is also conceivable that VSV and HIV-encoding plasmids transfected different cells in vivo, and that the VSV-G-induced cell to cell fusion resulted in a subsequent enhanced presentation of Gag antigen.

The enhancement in cytotoxic response observed following coinjection of Gag-encoding vector with VSV-encoding appears to operate by at least two different mechanisms, i.e. an activation of the immune system due to the nature of the VSV envelope itself and an increased processing of the secreted VSV-G-pseudotyped Gag particles.

The enhancement in antigen processing was further illustrated by in vitro experiments showing that exogenous presentation of Gag epitopes in APC was more efficient when Gag particles were pseudotyped with VSV-G. It is now well demonstrated that some exogenous antigens can be processed and presented to CD8+ T cells following the alternative class-I antigen presentation pathway APC (77, 113). In vitro studies showed that when Gag particles were pseudotyped with VSV-G envelope, the Gag protein enters both the MHC class I and the MHC class II processing pathways. By contrast, naked Gag particles only enter the MHC class II pathway and the derived epitopes are only recognized by CD4+ T cells.

Various successful strategies to prime MHC-I-restricted CD8⁺ CTL responses to exogenous antigen have been described to date. These include the parvovirus virus like particles (72, 106) the HIV-1 Gag core particle (64, 73), the hepatitis B surface antigen (104) and the yeast transposon-derived particle (82). Some of these approaches were combined with DNA-based immunization (75, 83, 120). Intramuscular administration of a DNA vaccine represents a simple and effective means of inducing both humoral and cellular immune responses including cytotoxic T cell responses (67). There are a number of strategies available to improve the potency of DNA vaccines. Such methods include i) DNA delivery systems such as cationic microparticles, that increase DNA transfer to APCs (109); ii) the inclusion of adjuvants, either as a gene or as a co-administered agent (48, 110); iii) the inclusion of immunostimulatory sequences such as CpG in the plasmid or vector modification to enhance antigen expression (75); iv) the inclusion of peptides that target the antigen to sites of immune reponse induction (63); v) codelivery of plasmids activating the death pathway (57, 103).

Direct injection into muscle cells induces synthesis, and in some cases secretion of recombinant protein (61, 91, 115). Targeting of the protein synthesized in the muscle to the dendritic cells operates through either cross-priming or secretion and capture of the DNA-encoded protein. The results are in agreement with the latter pathway for antigen capture, since a greater number of mice were obtained with anti-Gag specific cytotoxic activity and a greater efficiency in the cytotoxic response when production of Gag was achieved through coinjection of vectors encoding particles that were secreted and pseudotyped with a fusion-competent VSV-G envelope.

Because of the potential role of CTL in controlling HIV-1 infection (52, 80) and disease progression (90, 93), numerous approaches have been tested for activating the cellular immune response including for example non-pathogenic recombinant live vectors expressing HIV proteins, inactivated non-infectious virus particles and DNA vaccines (47, 48). Recently, an AIDS vaccine based on live attenuated recombinant VSV was shown to be effective in protecting macaques after challenge with a pathogenic virus (101). There is increasing evidence that both CD4+ and CD8+ subsets are probably required for strong CTL memory and protection against HIV-1 (95, 99, 102). HIV-1 Gag is one of the most conserved viral proteins and broad, cross-clade CTL responses recognizing conserved epitopes in HIV-1 Gag have been detected in HIV-1-infected individuals (50, 53, 68). Therefore, the induction of CTL and T-helper responses against conserved Gag epitopes via fusogenic envelope-mediated targeting of Gag particles to APC in vivo could be significant for the development of a safe and effective HIV-1 DNA vaccine.

In summary, CTLs detect viral infection by recognizing viral peptides bound to MHC-I. In most cells, peptides presented by MHC-I are classically thought to be derived from endogenously synthesized proteins. However, there is evidence in antigen presenting cells (APC), such as dendritic cells (DC), macrophage, and B cells, for a MHC-I-restricted pathway that presents peptides derived from extracellular antigens. DC and macrophage, are two major targets of HIV replication. The first steps of HIV life cycle include the entry of virions into the cytoplasm, and we have reported that incoming viral proteins may be degraded by the proteasome. This invention shows that APC present peptides derived from incoming virions. In B cells, MHC-I restricted exogenous presentation was observed with HIV(VSV) pseudotypes. It occurred efficiently in immature DC with HIV(VSV) pseudotypes and with both CXCR4- and CCR5-tropic viruses. The process was less efficient in macrophage than in DC and not detected in CD4+ lymphocytes. Exogenous presentation was not observed with virions lacking a fusogenic envelope, and therefore required a receptor-dependent transport of incoming virions into the cytosol.

Moreover, in vivo priming of cytotoxic T lymphocytes (CTL) by DNA injection predominantly occurs by antigen transfer from DNA-transfected cells to antigen presenting cells. A rational strategy for increasing DNA vaccine potency would be to use a delivery system that facilitates antigen uptake by antigen presenting cells. Exogenous antigen presentation through the MHC class I-restricted pathway of some viral antigens is increased after adequate virus-receptor interaction and the fusion of viral and cellular membrane. DNA-based immunization with plasmids coding for human immunodeficiency virus-type 1 (HIV-1) Gag particles pseudotyped with vesicular stomatitis virus glycoprotein (VSV-G) were used to generate Gag-specific CTL responses. The presence of the VSV-G encoding plasmid not only increased the number of mice displaying anti-Gag specific cytotoxic response, but also the efficiency of specific lysis. In vitro analysis of processing confirmed that exogenous presentation of Gag epitopes occurred much more efficiently when Gag particles were pseudotyped with the VSV-G envelope. This invention shows that the VSV-G-pseudotyped Gag particles not only entered the MHC class-II but also the MHC class-I processing pathway. In contrast, naked Gag particles entered the MHC class-II processing pathway only. Thus, the combined use of DNA-based immunization and nonreplicating pseudotyped virus for delivering HIV-1 antigen to the immune system in vivo could be considered in HIV-1 vaccine design.

The following references have been cited herein. The entire disclosure of each reference is relied upon and incorporated by reference herein.

1. Rock, K. L. A new foreign policy: MHC class I molecules monitor the outside world. Immunology today 17, 129-137 (1996).

2. Jondal, M., Schirmbeck, R. & Reimann, J. MHC class I-restricted CTL responses to exogenous antigens. Immunity 5, 295-302 (1996).

3. Yewdell, J. W. Mechanisms of exogenous antigen presentation by MHC class I molecules in vitro and in vivo: Implications for generating CD8+ T cell responses to infectious agents, tumors, transplants, and vaccines. Adv. in Immunol. 73, 1-77 (1999).

4. Lanzavecchia, A. Mechanisms of antigen uptake for presentation. Curr. Op. in Immunol. 8, 348-354 (1996).

5. Watts, C. Capture and processing of exogenous antigens for presentation on MHC molecules. Ann. Rev. Immunol. 15, 821-850 (1997).

6. Kovacsovics-Bankowski, M. & Rock, K. L. A phagosome-to cytosol pathway for exogenous antigens presented on MHC class I molecules. Science 267, 243-246 (1995).

7. Singh-Jasuja, H. et al. Cross-presentation of glycoprotein 96-associated antigens on major histocompatibility complex class I molecules requires receptor-mediated endocytosis. J. Exp. Med. 191, 1965-1974 (2000).

8. Castellino, F. et al. Receptor-mediated uptake of antigen/heat shock protein complexes results in major histocompatibility complex class 1 antigen presentation via two distinct processing pathways. J. Exp. Med. 191, 1957-1964 (2000).

9. Regnault, A. et al. Fcg receptor-mediated induction of dendritic cell maturation and major histocompatibility complex class I-restricted antigen presentation after immune complex internalization. J. Exp. Med. 189, 371-380 (1999).

10. Rodriguez, A., Regnault, A., Kleijmeer, M., Ricciardi-Castagnoli, P. & Amigorena, S. Selective transport of internalized antigens to the cytosol for MHC class I presentation in dendritic cells. Nature Cell Biol. 1, 362-368 (1999).

11. Gromme, M. et al. Recycling MHC class I molecules and endosomal peptide loading. Proc. Nat. Acad. Sci. (USA) 96,10326-10331 (1999).

12. Banchereau, J. & Steinman, R. M. Dendritic cells and the control of immunity. Nature 392, 245-252 (1998).

13. Albert, M. L., Sauter, B. & Bhardwaj, N. Dendritic cells acquire antigen from apoptotic cells and induce class I-restricted CTLs. Nature 392, 86-89 (1998).

14. Sigal, L. J., Crotty, S., Andino, R. & Rock, K. L. Cytotoxic T-cell immunity to virus-infected non-hematopoietic cells requires presentation of exogenous antigen. Nature 398, 77-80 (1999).

15. Yewdell, J. W., Bennink, J. R. & Hosaka, Y. Cells process exogenous proteins for recognition by cytotoxic T lymphocytes. Science 239, 637-640 (1988).

16. Reimann, J. & Schirmbeck, R. Alternative pathways for processing exogenous and endogenous antigens that can generate peptides for MHC class I-restricted presentation. Immunol. Rev. 172, 131-152 (1999).

17. Cameron, P., Pope, M., Granelli-Piperno, A. & Steinman, R. M. Dendritic cells and the replication of HIV-1. J. Leuk. Biol. 59, 158-171 (1996).

18. Knight, S. C. & Patterson, S. Bone marrow-derived dendritic cells, infection with human immunodeficiency virus, and immunopathology. Annu. Rev. Immunol. 15, 593-615 (1997).

19. Granelli-Piperno, A. et al. Efficient interaction of HIV-1 with purified dendritic cells via multiple chemokine coreceptors. J. Exp. Med. 184, 2433-2438 (1996).

20. Ayehunie, S. et al. Human immunodeficiency virus-1 entry into purified blood dendritic cells through CC and CXC chemokine coreceptors. Blood 90, 1379-1386 (1997).

21. Klagge, I. M. & Schneider-Schaulies, S. Virus interactions with dendritic cells. J. Gen. Virol. 80, 823-833 (1999).

22. Granelli-Piperno, A., Finkel, V., Delgado, E. & Steinman, R. M. Virus replication begins in dendritic cells during the transmission of HIV-1 from mature dendritic cells to T cells. Curr. Biol. 9, 21-29 (1999).

23. Geijtenbeek, T. B. et al. DC-SIGN, a dendritic cell-specific HIV-1-binding protein that enhances trans-infection of T cells. Cell 100, 587-597 (2000).

24. Lapham, C. K., Zaitseva, M. B., Lee, S., Romanstseva, T. & Golding, H. Fusion of monocytes and macrophages with HIV-1 correlates with biochemical properties of CXCR4 and CCR5. Nature Med. 5, 303-308 (1999).

25. Aiken, C. Pseudotyping HIV-1 by the glycoprotein of VSV targets HIV-1 entry to an endocytic pathway and suppresses both the requirement for Nef and the sensitivity to cyclosporin A. J. Virol. 71, 5871-5877 (1997).

26. Yang, O. O. et al. Efficient lysis of Human Immunodeficiency Virus type 1-infected cells by cytotoxic T lymphocytes. J. Virol. 70, 5799-5806 (1996).

27. Naldini, L. et al. In vivo gene delivery and stable transduction of non dividing cells by a lentiviral vector. Science 272, 263-267 (1996).

28. Rossio, J. L. et al. Inactivation of human immunodeficiency virus type 1 infectivity with preservation of conformational and functional integrity of virion surface proteins. J Virol 72, 7992-8001 (1998).

29. Arthur, L. O. et al. Chemical inactivation of retroviral infectivity by targeting nucleocapsid protein zinc fingers: a candidate SIV vaccine. AIDS Res. Hum. Retrov. 14, s311-s319 (1998).

30. Bergeron, L., Sullivan, N. & Sodroski, J. Target cell-specific determinants of membrane fusion within the Human Immunodeficiency virus type 1 gp120 third variable region and gp41 amino terminus. J. Virol. 66, 2389-2397 (1992).

31. Whitt, M. A., Zagouras, P., Crise, B. & Rose, J. K. A fusion-defective mutant of the Vesicular Stomatitis Virus glycoprotein. J. Virol. 64, 4907-4913 (1990).

32. Buseyne, F., Février, M., Garcia, S., Gougeon, M. L. & Rivière, Y. Dual function of a human immunodeficiency virus (HIV)-specific cytotoxic T-lymphocyte clone: inhibition of HIV replication by noncytolytic mechanisms and lysis of HIV-infected CD4+ cells. Virology 225, 248-253 (1996).

33. Piatak, M., Jr. et al. High levels of HIV-1 in plasma during all stages of infection determined by competitive PCR. Science 259,1749-1754 (1993).

34. Embreston, J. et al. Massive covert infection of helper T lymphocytes and macrophages by HIV during the incubation period of AIDS. Nature 362, 359-362 (1993).

35. Schwartz, O., Marechal, V., Friguet, B., Arenzana-Seisdedos, F. & Heard, J. M. Antiviral activity of the proteasome on incoming HIV-1. J. Virol. 72, 3845-3850 (1998).

36. Schwartz, O., Maréchal, V., Le Gall, S., Lemonnier, F. & Heard, J. M. Endocytosis of MHC-I molecules is induced by HIV-1 Nef. Nature Med. 2, 338-342 (1996).

37. Collins, K. L., Chen, B. K., Kalams, S. A., Walker, B. D. & Baltimore, D. HIV-1 Nef protein protects infected primary cells against killing by cytotoxic T lymphocytes. Nature 391, 397-401 (1998).

38. Pinto, L. A. et al. Env-specific cytotoxic T lymphocytes responses in HIV seronegative health care workers occupationally exposed to HIV-contaminated body fluids. J. Clin. Invest. 96, 867-876 (1995).

39. Rowland-Jones, S. et al. HIV-specific cytotoxic T-cells in HIV-exposed but uninfected Gambian women. Nature Med. 1, 59-64 (1995).

40. Rowland-Jones, S., Tan, R. & McMichael, A. Role of cellular immunity in protection against HIV infection. Adv. in Immunol. 65, 277-346 (1997).

41. Brander, C. & Walker, B. D. T lymphocyte responses in HIV-1 infection: implications for vaccine development. Curr. Op. in Immunol. 11, 451-459 (1999).

42. Goxe, B., Latour, N., Bartholeyns, J., Romet-Lemonne, J. L. & Chokri, M. Monocyte-derived dendritic cells: development of a cellular processor for clinical applications. Res. Immunol. 149, 643-646 (1998).

43. Buseyne, F. et al. Cross-clade-specific cytotoxic T lymphocytes in HIV-1-infected children. Virology 250, 316-324 (1998).

44. Tsomides, T.J. et al. Naturally processed viral peptides recognized by cytotoxic T lymphocytes on cells chronically infected by Human Immunodeficiency Virus type 1. J. Exp. Med. 180,1283-1293 (1994).

45. Czerkinsky, C. et al. Reverse ELISPOT assay for clonal analysis of cytokine production. I. Enumeration of gamma-interferon-secreting cells. J. Immunol. Meth. 110, 29-36 (1988).

46. Aiken, C. 1997. Pseudotyping human immunodeficiency virus type 1 (HIV-1) by the glycoprotein of vesicular stomatitis virus targets HIV-1 entry to an endocytic pathway and suppresses both the requirement for Nef and the sensitivity to cyclosporin A J Virol. 71:5871-7.

47. Amara, R. R., F. Villinger, J. D. Altman, S. L. Lydy, S. P. O'Neil, S. I. Staprans, D. C. Montefiori, Y. Xu, J. G. Herndon, L. S. Wyatt, M. A. Candido, N. L. Kozyr, P. L. Earl, J. M. Smith, H. L. Ma, B. D. Grimm, M. L. Hulsey, J. Miller, H. M. McClure, J. M. McNicholl, B. Moss, and H. L. Robinson 2001. Control of a mucosal challenge and prevention of AIDS by a multiprotein DNA/MVA vaccine Science. 292:69-74.

48. Barouch, D. H., A. Craiu, M. J. Kuroda, J. E. Schmitz, X. X. Zheng, S. Santra, J. D. Frost, G. R. Krivulka, M. A. Lifton, C. L. Crabbs, G. Heidecker, H. C. Perry, M. E. Davies, H. Xie, C. E. Nickerson, T. D. Steenbeke, C. I. Lord, D. C. Montefiori, T. B. Strom, J. W. Shiver, M. G. Lewis, and N. L. Letvin 2000. Augmentation of immune responses to HIV-1 and simian immunodeficiency virus DNA vaccines by IL-2/lg plasmid administration in rhesus monkeys Proc Natl Acad Sci U S A. 97:4192-7.

49. Bennett, S. R., F. R. Carbone, F. Karamalis, J. F. Miller, and W. R. Heath 1997. Induction of a CD8+ cytotoxic T lymphocyte response by cross-priming requires cognate CD4+ T cell help J Exp Med. 186:65-70.

50. Betts, M. R., J. Krowka, C. Santamaria, K. Balsamo, F. Gao, G. Mulundu, C. Luo, N. N'Gandu, H. Sheppard, B. H. Hahn, S. Allen, and J. A. Frelinger 1997. Cross-clade human immunodeficiency virus (HIV)-specific cytotoxic T-lymphocyte responses in HIV-infected Zambians J Virol. 71:8908-11.

51. Bevan, M. J. 1976. Cross-priming for a secondary cytotoxic response to minor H antigens with H-2 congenic cells which do not cross-react in the cytotoxic assay J Exp Med. 143:1283-8.

52. Borrow, P., H. Lewicki, B. H. Hahn, G. M. Shaw, and M. B. Oldstone 1994. Virus-specific CD8+ cytotoxic T-lymphocyte activity associated with control of viremia in primary human immunodeficiency virus type 1 infection J Virol. 68:6103-10.

53. Buseyne, F., M. L. Chaix, C. Rouzioux, S. Blanche, and Y. Riviere 2001. Patient-specific cytotoxic T-lymphocyte cross-recognition of naturally occurring variants of a human immunodeficiency virus type 1 (HIV-1) p24gag epitope by HIV-1-infected children J Virol. 75:4941-6.

54. Buseyne, F., M. Février, S. Garcia, M. L. Gougeon, and Y. Rivière 1996. Dual functions of a human immunodeficiency virus (HIV) specific cytotoxic T-lymphocytes (CTL) clone: inhibition of HIV replication by non-cytolytic mechanisms and lysis of HIV-infected CD4+ cells. Virology. 225:248-253.

55. Buseyne, F., S. Le Gall, C. Boccaccio, J. P. Abastado, J. D. Lifson, L. O. Arthur, Y. Riviere, J. M. Heard, and 0. Schwartz 2001. MHC-I-restricted presentation of HIV-1 virion antigens without viral replication Nat Med. 7:344-9.

56. Chan, K., D. J. Lee, A. Schubert, C. M. Tang, B. Crain, S. P. Schoenberger, and M. Corr 2001. The roles of MHC class II, CD40, and B7 costimulation in CTL induction by plasmid DNA J Immunol. 166:3061-6.

57. Chattergoon, M. A., J. J. Kim, J. S. Yang, T. M. Robinson, D. J. Lee, T. Dentchev, D. M. Wilson, V. Ayyavoo, and D. B. Weiner 2000. Targeted antigen delivery to antigen-presenting cells including dendritic cells by engineered Fas-mediated apoptosis Nat Biotechnol. 18:974-9.

58. Condon, C., S. C. Watkins, C. M. Celluzzi, K. Thompson, and L. D. Falo, Jr. 1996. DNA-based immunization by in vivo transfection of dendritic cells Nature Med. 2:1122-8.

59. Corr, M., D. J. Lee, D. A. Carson, and H. Tighe 1996. Gene vaccination with naked plasmid DNA: mechanism of CTL priming J. Exp. Med. 184:1555-1560.

60. Corr, M., A. von Damm, D. J. Lee, and H. Tighe 1999. In vivo priming by DNA injection occurs predominantly by antigen transfer J Immunol. 163:4721-7.

61. Davis, H. L., M.-L. Michel, and R. G. Whalen 1993. DNA based immunization for hepatitis B induces continuous secretion of antigen and high levels of circulating antibody Hum. Molec. Genet. 2:1847-1851.

62. Davis, H. L., R. G. Whalen, and B. A. Demeneix 1993. Direct gene transfer into skeletal muscle in vivo: factors affecting efficiency of transfer and stability of expression Hum. Gene Ther. 4:151-159.

63. Deliyannis, G., J. S. Boyle, J. L. Brady, L. E. Brown, and A. M. Lew 2000. A fusion DNA vaccine that targets antigen-presenting cells increases protection from viral challenge Proc Natl Acad Sci U S A. 97:6676-80.

64. Deml, L., R. Schirmbeck, J. Reimann, H. Wolf, and R. Wagner 1997. Recombinant human immunodeficiency Pr55gag virus-like particles presenting chimeric envelope glycoproteins induce cytotoxic T-cells and neutralizing antibodies Virology. 235:26-39.

65. Doe, B., M. Selby, S. Barnett, J. Baenziger, and C. M. Walker 1996. Induction of cytotoxic T lymphocytes by intramuscular immunization with plasmid DNA is facilitated by bone marrow-derived cells Proc. Natl. Acad. Sci. USA. 93:8578-8583.

66. Donnelly, J. J., M. A. Liu, and J. B. Ulmer 2000. Antigen presentation and DNA vaccines Am J Respir Crit Care Med. 162:S190-3.

67. Donnelly, J. J., J. B. Ulmer, J. W. Shiver, and M. A. Liu 1997. DNA vaccines Annu. Rev. Immunol. 15:617-648.

68. Durali, D., J. Morvan, F. Letourneur, D. Schmitt, N. Guegan, M. Dalod, S. Saragosti, D. Sicard, J. P. Levy, and E. Gomard 1998. Cross-reactions between the cytotoxic T-lymphocyte responses of human immunodeficiency virus-infected African and European patients J Virol. 72:3547-53.

69. Fu, T. M., J. B. Ulmer, M. J. Caulfield, R. R. Deck, A. Friedman, S. Wang, X. Liu, J. J. Donnelly, and M. A. Liu 1997. Priming of cytotoxic T lymphocytes by DNA vaccines: requirement for professional antigen presenting cells and evidence for antigen transfer from myocytes Molec. Med. 3:362-371.

70. Germain, R. N. 1994. MHC-dependent antigen processing and peptide presentation: providing ligands for T lymphocyte activation Cell. 76:287-99.

71. Granelli-Piperno, A., L. Zhong, P. Haslett, J. Jacobson, and R. M. Steinman 2000. Dendritic cells, infected with vesicular stomatitis virus-pseudotyped HIV-1, present viral antigens to CD4+ and CD8+ T cells from HIV-1-infected individuals J Immunol. 165:6620-6.

72. Greenstone, H. L., J. D. Nieland, K. E. de Visser, M. L. De Bruijn, R. Kirnbauer, R. B. Roden, D. R. Lowy, W. M. Kast, and J. T. Schiller 1998. Chimeric papillomavirus virus-like particles elicit antitumor immunity against the E7 oncoprotein in an HPV16 tumor model Proc Natl Acad Sci U S A. 95:1800-5.

73. Griffiths, J. C., S. J. Harris, G. T. Layton, E. L. Berrie, T. J. French, N. R. Burns, S. E. Adams, and A. J. Kingsman 1993. Hybrid human immunodeficiency virus Gag particles as an antigen carrier system: induction of cytotoxic T-cell and humoral responses by a Gag:V3 fusion J Virol. 67:3191-8.

74. Huang, A. Y., P. Golumbek, M. Ahmadzadeh, E. Jaffee, D. Pardoll, and H. Levitsky 1994. Role of bone marrow-derived cells in presenting MHC class I-restricted tumor antigens Science. 264:961-5.

75. Huang, Y., W. P. Kong, and G. J. Nabel 2001. Human immunodeficiency virus type 1-specific immunity after genetic immunization is enhanced by modification of Gag and Pol expression J Virol. 75:4947-51.

76. Iwasaki, A., B. J. Stiernholm, A. K. Chan, N. L. Berinstein, and B. H. Barber 1997. Enhanced CTL responses mediated by plasmid DNA immunogens encoding costimulatory molecules and cytokines J Immunol. 158:4591-601.

77. Jondal, M., R. Schirmbeck, and J. Reimann 1996. MHC class I-restricted CTL responses to exogenous antigens Immunity. 5:295-302.

78. Kim, J. J., M. L. Bagarazzi, N. Trivedi, Y. Hu, K. Kazahaya, D. M. Wilson, R. Ciccarelli, M. A. Chattergoon, K. Dang, S. Mahalingam, A. A. Chalian, M. G. Agadjanyan, J. D. Boyer, B. Wang, and D. B. Weiner 1997. Engineering of in vivo immune responses to DNA immunization via codelivery of costimulatory molecule genes Nat Biotechnol. 15:641-6.

79. Kim, J. J., J. S. Yang, T. C. VanCott, D. J. Lee, K. H. Manson, M. S. Wyand, J. D. Boyer, K. E. Ugen, and D. B. Weiner 2000. Modulation of antigen-specific humoral responses in rhesus macaques by using cytokine cDNAs as DNA vaccine adjuvants J Virol. 74:3427-9.

80. Koup, R. A., J. T. Safrit, Y. Cao, C. A. Andrews, G. McLeod, W. Borkowsky, C. Farthing, and D. D. Ho 1994. Temporal association of cellular immune responses with the initial control of viremia in primary human immunodeficiency virus type 1 syndrome J Virol. 68:4650-5.

81. Kurts, C., J. F. Miller, R. M. Subramaniam, F. R. Carbone, and W. R. Heath 1998. Major histocompatibility complex class I-restricted cross-presentation is biased towards high dose antigens and those released during cellular destruction J Exp Med. 188:409-14.

82. Layton, G. T., S. J. Harris, A. J. Gearing, M. Hill-Perkins, J. S. Cole, J. C. Griffiths, N. R. Burns, A. J. Kingsman, and S. E. Adams 1993. Induction of HIV-specific cytotoxic T lymphocytes in vivo with hybrid HIV-1 V3:Ty-virus-like particles J Immunol. 151:1097-107.

83. Le Borgne, S., M. Mancini, R. Le Grand, M. Schleef, D. Dormont, P. Tiollais, Y. Riviere, and M. L. Michel 1998. In vivo induction of specific cytotoxic T lymphocytes in mice and rhesus macaques immunized with DNA vector encoding an HIV epitope fused with hepatitis B surface antigen Virology. 240:304-15.

84. Lewis, P. J., H. van Drunen Littel-van den, and L. A. Babiuk 1999. Altering the cellular location of an antigen expressed by a DNA-based vaccine modulates the immune response J Virol. 73:10214-23.

85. Loirat, D., Z. Li, M. Mancini, P. Tiollais, D. Paulin, and M. L. Michel 1999. Muscle-specific expression of hepatitis B surface antigen: no effect on DNA-raised immune responses Virology. 260:74-83.

86. Lu, Z., L. Yuan, X. Zhou, E. Sotomayor, H. I. Levitsky, and D. M. Pardoll 2000. CD40-independent pathways of T cell help for priming of CD8(+) cytotoxic T lymphocytes J Exp Med. 191:541-50.

87. Mancini, M., H. L. Davis, P. Tiollais, and M.-L. Michel 1996. DNA-based immunization against the envelope proteins of the hepatitis B virus J. Biotechnol. 44:47-57.

88. Mancini, M., M. Hadchouel, P. Tiollais, and M. L. Michel 1998. Regulation of hepatitis B virus mRNA expression in a hepatitis B surface antigen transgenic mouse model by IFN-gamma-secreting T cells after DNA-based immunization J. Immunol. 161:5564-5570.

89. Marechal, V., F. Clavel, J. M. Heard, and O. Schwartz 1998. Cytosolic Gag p24 as an index of productive entry of human immunodeficiency virus type 1 J Virol. 72:2208-12.

90. Musey, L., J. Hughes, T. Schacker, T. Shea, L. Corey, and M. J. McElrath 1997. Cytotoxic-T-cell responses, viral load, and disease progression in early human immunodeficiency virus type 1 infection N Engl J Med. 337:1267-74.

91. Naffakh, N., C. Pinset, D. Montarras, Z. LI, D. Paulin, O. Danos, and J.-M. Heard 1996. Long-term secretion of therapeutic proteins from genetically modified skeletal muscles Hum. Gene Ther. 7:11-21.

92. Naldini, L., U. Blomer, P. Gallay, D. Ory, R. Mulligan, F. H. Gage, I. M. Verma, and D. Trono 1996. In vivo gene delivery and stable transduction of nondividing cells by a lentiviral vector Science. 272:263-7.

93. Ogg, G. S., X. Jin, S. Bonhoeffer, P. R. Dunbar, M. A. Nowak, S. Monard, J. P. Segal, Y. Cao, S. L. Rowland-Jones, V. Cerundolo, A. Hurley, M. Markowitz, D. D. Ho, D. F. Nixon, and A. J. McMichael 1998. Quantitation of HIV-1-specific cytotoxic T lymphocytes and plasma load of viral RNA Science. 279:2103-6.

94. Okimoto, T., T. Friedmann, and A. Miyanohara 2001. Vsv-g envelope glycoprotein forms complexes with plasmid dna and mlv retrovirus-like particles in cell-free conditions and enhances dna transfection Mol Ther. 4:232-8.

95. Pitcher, C. J., C. Quittner, D. M. Peterson, M. Connors, R. A. Koup, V. C. Maino, and L. J. Picker 1999. HIV-1-specific CD4+ T cells are detectable in most individuals with active HIV-1 infection, but decline with prolonged viral suppression Nat Med. 5:518-25.

96. Rautmann, G., M. P. Kieny, R. Brandely, K. Dott, M. Girard, L. Montagnier, and J. P. Lecocq 1989. HIV-1 core proteins expressed from recombinant vaccinia viruses AIDS Res Hum Retroviruses. 5:147-57.

97. Reimann, J., and R. Schirmbeck 1999. Alternative pathways for processing exogenous and endogenous antigens that can generate peptides for MHC class I-restricted presentation Immunol Rev. 172:131-52.

98. Ridge, J. P., F. Di Rosa, and P. Matzinger 1998. A conditioned dendritic cell can be a temporal bridge between a CD4+T- helper and a T-killer cell Nature. 393:474-8.

99. Riviere, Y., M. B. McChesney, F. Porrot, F. Tanneau-Salvadori, P. Sansonetti, O. Lopez, G. Pialoux, V. Feuillie, M. Mollereau, S. Chamaret, and et al. 1995. Gag-specific cytotoxic responses to HIV type 1 are associated with a decreased risk of progression to AIDS-related complex or AIDS AIDS Research & Human Retroviruses. 11:903-7.

100. Robinson, H. L., and C. Torres 1997. DNA vaccines Seminars in Immunology. 9:271-283.

101. Rose, N. F., P. A. Marx, A. Luckay, D. F. Nixon, W. J. Moretto, S. M. Donahoe, D. Montefiori, A. Roberts, L. Buonocore, and J. K. Rose 2001. An effective aids vaccine based on live attenuated vesicular stomatitis virus recombinants Cell. 106:539-49.

102. Rosenberg, E. S., J. M. Billingsley, A. M. Caliendo, S. L. Boswell, P. E. Sax, S. A. Kalams, and B. D. Walker 1997. Vigourous HIV-1-specific CD4+ T cell responses associated with control of viremia Science. 278:1447-1450.

103. Sasaki, S., R. R. Amara, A. E. Oran, J. M. Smith, and H. L. Robinson 2001. Apoptosis-mediated enhancement of DNA-raised immune responses by mutant caspases Nat Biotechnol. 19:543-7.

104. Schirmbeck, R., K. Melber, A. Kuhrober, Z. A. Janowicz, and J. Reimann 1994. Immunization with soluble hepatitis B virus surface protein elicits murine H-2 class I-restricted CD8+ cytotoxic T lymphocyte responses in vivo J. Immunol. 152:1110-1119.

105. Schoenberger, S. P., R. E. Toes, E. I. van der Voort, R. Offringa, and C. J. Melief 1998. T-cell help for cytotoxic T lymphocytes is mediated by CD40-CD40L interactions Nature. 393:480-3.

106. Sedlik, C., M. Saron, J. Sarraseca, I. Casal, and C. Leclerc 1997. Recombinant parvovirus-like particles as an antigen carrier: a novel nonreplicative exogenous antigen to elicit protective antiviral cytotoxic T cells Proc Natl Acad Sci U S A. 94:7503-8.

107. Selby, M. J., B. Doe, and C. M. Walker 1997. Virus-specific cytotoxic T-lymphocyte activity elicited by coimmunization with human immunodeficiency virus type 1 genes regulated by the bacteriophage T7 promoter and T7 RNA polymerase protein J Virol. 71:7827-31.

108. Sharma, S., A. Miyanohara, and T. Friedmann 2000. Separable mechanisms of attachment and cell uptake during retrovirus infection J Virol. 74:10790-5.

109. Singh, M., M. Briones, G. Ott, and D. O'Hagan 2000. Cationic microparticles: A potent delivery system for DNA vaccines Proc Natl Acad Sci U S A. 97:811-6.

110. Ulmer, J. B., C. M. DeWitt, M. Chastain, A. Friedman, J. J. Donnelly, W. L. McClements, M. J. Caulfield, K. E. Bohannon, D. B. Volkin, and R. K. Evans 1999. Enhancement of DNA vaccine potency using conventional aluminum adjuvants Vaccine. 18:18-28.

111. Wang, B., C. C. Norbury, R. Greenwood, J. R. Bennink, J. W. Yewdell, and J. A. Frelinger 2001. Multiple paths for activation of naive cd8(+) t cells: cd4-independent help J Immunol. 167:1283-9.

112. Wang, R., D. L. Doolan, T. P. Le, R. C. Hedstrom, K. M. Coonan, Y. Charoenvit, T. R. Jones, P. Hobart, M. Margalith, J. Ng, W. R. Weiss, M. Sedegah, C. de Taisne, J. A. Norman, and S. L. Hoffman 1998. Induction of antigen-specific cytotoxic T lymphocytes in humans by a malaria DNA vaccine Science. 282:476-480.

113. Watts, C. 1997. Capture and processing of exogenous antigens for presentation on MHC molecules Annu Rev Immunol. 15:821-50.

114. Whitt, M. A., P. Zagouras, B. Crise, and J. K. Rose 1990. A fusion-defective mutant of the Vesicular Stomatitis Virus glycoprotein. J. Virol. 64:4907-4913.

115. Wolff, J. A., J. J. Ludtke, G. Acsadi, P. Williams, and A. Jani 1992. Long-term persistence of plasmid DNA and foreign gene expression in mouse muscle Hum Mol Genet. 1:363-9.

116. Xiang, Z., and H. C. Ertl 1995. Manipulation of the immune response to a plasmid-encoded viral antigen by coinoculation with plasmids expressing cytokines Immunity. 2:129-35.

117. Yee, J. K., A. Miyanohara, P. LaPorte, K. Bouic, J. C. Burns, and T. Friedmann 1994. A general method for the generation of high-titer, pantropic retroviral vectors: highly efficient infection of primary hepatocytes Proc Nati Acad Sci U S A. 91:9564-8.

118. Yewdell, J. W., C. C. Norbury, and J. R. Bennink 1999. Mechanisms of exogenous antigen presentation by MHC class I molecules in vitro and in vivo: implications for generating CD8+ T cell responses to infectious agents, tumors, transplants, and vaccines Adv Immunol. 73:1-77.

119. Zufferey, R., D. Nagy, R. J. Mandel, L. Naldini, and D. Trono 1997. Multiply attenuated lentiviral vector achieves efficient gene delivery in vivo Nat Biotechnol. 15:871-5.

120. zur Megede, J., M. C. Chen, B. Doe, M. Schaefer, C. E. Greer, M. Selby, G. R. Otten, and S. W. Barnett 2000. Increased expression and immunogenicity of sequence-modified human immunodeficiency virus type 1 gag gene J Virol. 74:2628-35.

Acknowledgements.

This work was supported in part by Federal funds from the National Cancer Institute, National Institutes of Health, under Contract No. NO1-CO-56000.

What is claimed is:

1. An immunogenic composition capable of inducing a cytotoxic response in vitro or in vivo against a viral disease through a MHC-1 restricted exogenous antigen presentation pathway without requiring viral replication, containing at least one of the compounds:
(A) a first plasmid containing a polynucleotide corresponding to the entire or a part of the viral genome and a second plasmid comprising in an insert containing a polynucleotide coding for a viral envelope (a part of the envelope or a surface protein) and being under the control of a promoter, said plasmids being selected for their fusogenic properties when binding to antigen presentation cells, and for inducing a cytotoxic response through a MHC-1 restricted exogenous antigen presentation pathway;
(B) a plasmid comprising a polynucleotide coding for the entire or a part of the virus genome and contains an insert containing a polynucleotide coding for a viral envelope (or a part of the envelope or a surface protein), and being under the control of a promoter said plasmid expressing viral particles being selected for their fusogenic non-replicative properties, and for inducing a cytotoxic response afer a CMH-2 restricted exogenous antigen presentation pathway;
(C) a virus with intact fusogenic capacities, but whose infectious capacities have been inactivated or attenuated; and
(D) viral particles obtained by the purification of a cell culture supernatant.

2. An immunogenic composition according to claim 1 wherein the viral particles obtained by the purification of a cell culture supernatant are prepared by transfecting producing cells (for example, HeLa, 293) with the plasmids according to claim 1 and purifying the supernatant, or by infecting antigen presenting cells with an HIV virus, purifying the supernatant, and inactivating or attenuating the infectious capacity of the virus.

3. A vaccinating composition containing the immunogenic composition according to claim 2 in association with a pharmaceutically acceptable vehicle.

4. A vaccinating composition containing the immunogenic composition according to claim 2 in association with another vaccine.

5. A vaccinating composition containing the immunogenic composition according to claim 2 wherein the composition is obtained by the process of claim 16.

6. A process of treatment of a eukaryotic host suffering from a viral pathology comprising administering a plasmid comprising a polynucleotide coding for the entire or a part of the virus genome and containing an insert containing a polynucleotide coding for a viral envelope (or a part of the envelope or a surface protein), and being under the control of a promoter, said plasmid expressing viral particles being selected for its fusogenic, non-replicative properties, and for inducing a cytotoxic response after a CMH-1 restricted exogenous antigen presentation pathway.

7. A process of treatment of a eukaryotic host suffering from a viral pathology comprising coadministering a first plasmid comprising the entire or a part of the virus genome and a second plasmid comprising an insert containing a polynucleotide coding for a viral envelope (a part of the envelope or a surface protein) and being under the control of a promoter, said plasmid being selected for its fusogenic properties, and for inducing a cytotoxic response after an exogenous antigen presentation which is MHC-1 restricted.

8. A process of treatment according to claim 6 or 7, wherein the virus is an human or animal retrovirus.

9. A process of treatment according to claim 6 or 7, wherein the virus is HIV-1, HIV-2, SIV, FeLV, or FIV.

10. A process of treatment according to claim 6 or 7, wherein that the host is a mammal.

11. A process of treatment according to claim 6 or 7, wherein the host is a mouse.

12. A process of stimulation in vivo of cytotoxic lymphocytes through an MHC-1 restricted exogenous antigen presentation pathway without requiring viral replication, comprising:
(A) administration of the plasmids contained in the immunogenic composition according to claim 1 or 2 to the host according to claim 10;
(B) optionally the cytotoxic T cells obtained after the step A above are tested in a cytotoxic test comprising:
(i) the incubation of an organ or a biologic fluid of the host containing cytotoxic T cells of the host with a synthetic peptide which sequence is encoded by a viral genome contained partly in the first or the second plasmid; or
(ii) the use of target cells with the same HLA haplotype as the host or a compatible HLA haplotype, said target cell being incubated with a synthetic peptide which sequence is a part of the sequence of an HIV-genome.

13. A process of stimulation in vivo of cytotoxic lymphocytes through an MHC-1 restricted exogenous antigen presentation pathway without requiring viral replication, comprising:
(A) administration of viral particles obtained by supernatant purification according to claim 2;
(B) optionally the cytotoxic T cells obtained after step A above are tested in a cytotoxic test comprising:
(i) the incubating of an organ or a biologic fluid of the host containing cytotoxic T cells of the host with a synthetic peptide which sequence is encoded by the genome contained partly in the first or the second plasmid; or
(ii) the use of target cells with the same HLA haplotype as the host or a compatible HLA haplotype, said target cells being incubated with a synthetic peptide which sequence is a part of an HIV genome.

14. A process of stimulation in vivo of cytotoxic lymphocytes by exogenous antigen presentation without viral replication comprising:
(A) administration of an HIV virus which infectious capacities have been inactivated or attenuated, but whose fusogenic capacities are intact according to claim 2;
(B) optionally the cytotoxic T cells obtained after the step A above are tested in a cytotoxic test comprising:
(i) the incubation of an organ or a biologic fluid of the host containing cytotoxic T cells of the host with a synthetic peptide which sequence is encoded by the viral genome contained partly in the first or the second plasmid; or
(ii) the use of target cells with the same HLA haplotype as the host or a compatible HLA haplotype, said target cell being incubated with a synthetic peptide which sequence is a part of the sequence of an HIV genome.

15. A process of treatment of an eukaryotic host suffering from a viral pathology, wherein antigen presenting cells are treated with the immunogenic composition of claims 1 to 4 then administrated back to the mammal after incubation.

16. A process of screening a composition, which is capable of inducing against a viral pathology a cytotoxic response in vitro or in vivo by exogenous antigen presentation without viral replication, wherein the cytotoxic activity of said composition is determined by the process according to claims 12 to 14.

17. A method of determining cytotoxic T lymphocyte (CTL) reponse to an antigen, wherein the method comprises:
providing viral particles containing the antigen and having a fusogenic envelope membrane;
targeting the viral particles into professional antigen presenting cells (APCs) by binding of the viral particles to the plasma membranes of the APCs and uptake of the viral particles by the APCs following fusion of the fusogenic envelope membranes of the viral particles with the plasma membranes of the APCs, which is followed by MHC-I-restricted presentation of the antigen by the APCs without viral replication or de novo, in situ synthesis of the antigen in the APCs;
contacting the resulting transduced APCs with CTLs that recognize MHC-I-restricted antigen; and
determining cell cytotoxicity resulting from said contact.

18. The method as claimed in claim 17, wherein the antigen is an HIV-1 antigen and the viral particles are attenuated or inactivated HIV-1 viral particles.
